SEQUENCE LISTING (21 sequences)

SEQ ID NO: 1 - DNA, 17572 bp, Bombyx mori
atgagagtcaaaacctttgtgatcttgtgctgcgctctgcaggtgagttaattattttactattatttcagaaggtggccagacgatatcacgggccacctgataataagtggtcgccaaaacgcacagatatcgtaaattgtgccatttgatttgtcacgcccgggggggctacggaataaactacatttatttatttaaaaaatgaaccttagattatgtaacttgtgatttatttgcgtcaaaagtaggcaagatgaatctatgtaaatacctgggcagacttgcaatatcctatttcaccggtaaatcagcattgcaatatgcaatgcatattcaacaatatgtaaaacaattcgtaaagcatcattagaaaatagacgaaagaaattgcataaaattataaccgcattattaatttattatgatatctattaacaattgctattgcctttttttcgcaaattataatcattttcataacctcgaggtagcattctgttacattttaatacattggtatgtgattataacacgagctgcccactgagtttctcgccagatcttctcagtgggtcgcgttaccgatcacgtgatagattctatgaagcactgctcttgttagggctagtgttagcaaattctttcaggttgagtctgagagctcacctacccatcggagcgtagctggaataggctaccagctaataggtagggaaaacaaagctcgaaacaagctcaagtaataacaacataatgtgaccataaaatctcgtggtgtatgagatacaattatgtactttcccacaaatgtttacataattagaatgttgttcaacttgcctaacgccccagctagaacattcaattattactattaccactactaaggcagtatgtcctaactcgttccagatcagcgctaacttcgattgaatgtgcgaaatttatagctcaatattttagcacttatcgtattgatttaagaaaaaattgttaacattttgtttcagtatgtcgcttatacaaatgcaaacatcaatgattttgatgaggactattttgggagtgatgtcactgtccaaagtagtaatacaacagatgaaataattagagatgcatctggggcagttatcgaagaacaaattacaactaaaaaaatgcaacggaaaaataaaaaccatggaatacttggaaaaaatgaaaaaatgatcaagacgttcgttataaccacggattccgacggtaacgagtccattgtagaggaagatgtgctcatgaagacactttccgatggtactgttgctcaaagttatgttgctgctgatgcgggagcatattctcagagcgggccatacgtatcaaacagtggatacagcactcatcaaggatatacgagcgatttcagcactagtgctgcagtcggtgcaggagctggtgcaggtgctgccgctggttctggtgcgggtgccggagctggttatggagctgcttctggtgctggtgccggtgctggggctggtgccggagctggttatggaactggtgcaggtgcaggtgccggagctggttatggagctggtgcaggtgcaggtgccggagctggttatggggctggtgcaggtgcaggtgccggagctggttatggagctggtgcaggtgcaggtgccggagctggttatggggctggtgcaggtgcaggtgccggagctggttatggagctggtgcgggtgccggtgccggggctggttatggagctgcctctggtgctggtgctggcgctgggtacggacaaggagtaggaagcggagctgcttctggagctggtgcaggtgcaggagcaggttctgccgctggttctggggcaggtgccggtgctggtaccggtgctggtgcaggttacggagctggtgcaggtgccggtgccggagctggttatggagctgcctctggtactggagcaggttatggagctggtgccggagctggttacggaggtgcctctggtgctggtgctggtgccggtgctggggctggagccggtgctggtgcaggttatggaactggcgctggatacggagcaggagccggagcaggagccggagcaggagctggtgctggatacggagcaggagctggtgctggatacggagcaggatatggagtaggagctggtgctggatacggagcaggatacggagcaggagctggaagcggagctgcctctggtgctggttcaggtgccggtgctggttcaggtgccggtgctggttcaggtgccggtgctggttcaggtgccggtgccggttcaggtgctggtgctggttcaggtgctggtgctggttcaggtgctggtgcaggttcaggtgctggtgctggttcaggtactggtgctggttcaggagctggtgctggatacggagcaggagctggtgctggatacggagcaggagcaggaagtggagctgcctctggtgccggtgctggttcaggtgcaggtgctggttcaggtgctggtgctggttcaggtgctggtgctggttcaggtgctggtgctggttcaggagctggtgctggatacggagcaggagctggtgctggatacggagcaggagctggtgctggatacggagcaggagctggcgttggatacggagcaggagctggaagcggagctgcctctggtgctggtgctggttcaggagccggtgctggttcaggtgctggtgctggttcaggtgctggtgctggttcaggtgctggtgctggttcaggtgccggtgctggttcaggtgctggtgctggttcaggagctggtgctggttcaggtgctggtgctggttcaggagctggagttggatacggagcaggagttggtgctggatacggagcaggatatggagcaggagctggtgctggatacggagcaggagcaggaagcggagctgcctctggtgctggtgccggtgctggagctggtgcaggaacaggctcttctggatttggaccatatgtagcaaatggcggatatagcagaagtgatggctacgaatacgcttggtcgtctgactttggaactggaagcggagctggtgctggttcaggtgctggtgctggttcaggtgctggcgctggctcaggtgctggtgctggttcaggtgctggtgctggttcaggagctggagctggatacggagcaggagttggtgttggatacggagcaggatatggagcaggagctggtgctggatacggagcaggagcaggaagcggagctgcctctggtgccggtgctggttcaggtgctggtgctggttcaggtgccggtgctggttcaggtgctggtgctggttcaggtgccggtgctggttcaggtgctggtgctggctcaggtgccggtgctggttcaggtgctggtgctggttcaggtgccggtgctggttcaggtgctggtgctggttcaggtgctggtgttggctcaggtgctggtgctggttcaggtgctggtgctggcgttggatacggagcaggagctggcgttggatacggagcaggagctggaagcggagctgcctctggtgctggtgctggttcaggagccggtgctggttcaggtgctggtgctggttcaggtgctggtgctggttcaggtgctggtgctggttcaggtgccggtgctggttcaggtgctggtgctggttcaggagctggtgctggttcaggtgctggtgctggttcaggagctggagttggatacggagcaggagttggtgctggatacggagcaggatatggagcaggagctggtgctggatacggagcaggagcaggaagcggagctgcctcaggtgccggtgctggttcaggtgctggtgctggttcaggtgccggtgctggttcaggtgctggtgctggttcaggtgctggtgctggttcaggtgctggtgctggttcaggtgctggtgctggttcaggtgctggtgctggttcaggtgctggtgctggctcaggtgctggagctggttcaggtgctggtgctggttcaggagctggagctggatacggagcaggagctggtgctggatacggagcaggatatggagcaggagctggtgctggatacggagcaggagctggaagcggagctgcctctggtgctggttcaggtgccggtgctggttcaggtgccggtgctggtgctggttcaggtgctggtgctggttcaggtgctggtgctggttcaggtgctggtgctggctcaggtgctggagctggttcaggtgctggtgctggttcaggagctggagctggatacggagcaggagttggtgctggatacggagcaggatatggagcaggagctggtgctggatacggagcaggagcaggaagcggagctgcctctggtgccggtgctggttcaggtgctggagctggttcaggtgccggtgctggttcaggtgctggtgctggttcaggagctggtgctggttcaggagctggtgctggttcaggtgctggtgctggttcaggagctggagttggatacggagcaggatatggagcaggagctggtgctggatacggagcaggagcaggaagcggagctgcctctggtgctggtgccggtgctggagctggtgcaggaacaggctcttctggatttggaccatatgtagcacatggcggatatagcggctacgaatacgcttggtcgtcagaatctgactttggaactggaagcggagctggtgctggttcaggtgctggtgctggttcaggtgctggtgctggctcaggtgctggtgctggttcaggagctggatacggagcaggagttggtgctggatacggagcaggatatggagcaggagctggtgctggatacggagcaggagcaggaagcggagctggctcaggtgctggtgctggttcaggagctggagctggttcaggtgccggtgctggttcaggtgctggtgctggttcaggagctggtgctggttcaggtgctggtgctggttcaggagctggtgctggttcaggtgccggtgctggttcaggtgctggtgctggatacggagcaggatatggagcaggagctggtgctggatacggagcaggagcaggaagcggagctggctcaggtgctggtgctggttcaggtgctggtgctggttcaggagctggagctggttcaggtgccggtgctggttcaggtgctggtgctggttcaggtgctggtgctggctcaggtgctggtgctggttcaggagctggagctggatacggagcaggagttggtgctggatacggagcaggatatggagcaggagctggtgctggatacggagcaggagcaggaagcggagctggctcaggtgctggtgctggttcaggagctggagctggttcaggtgctggtgctggttcaggtgccggtgttggttcaggtgctggtgctggttcaggagctggtgctggttcaggtgccggtgctggttcaggtgctggtgctggatacggagcaggatatggagcaggagctggtgctggatacggagcaggagcaggaagcggagctggctcaggtgctggtgctggttcaggtgctggtgctggttcaggagctggagctggttcaggtgccggtgctggttcaggtgctggtgctggttcaggagctggtgctggttcaggtgctggtgctggttcaggagctggagttggatacggagcaggagttggtgctggatacggagcaggatatggagcaggagctggtgctggatacggagcaggagcaggaagcggagctgcctctggtgctggtgccggtgctggagctggtgcaggaacaggctcttctggatttggaccatatgtagcaaatggcggatatagcggctacgaatacgcttggtcgtcagaatctgactttggaactggaagcggagctggtgctggttcaggtgctggtgctggttcaggtgctggtgctggctcaggtgctggtgctggttcaggagctggagctggatacggagcaggatatggagcaggagctggtgctggatacggagcaggagcaggaagcggagctggctcaggtgctggtgctggttcaggagctggagctggttcaggtgccggtgctggttcaggtgctggtgctggttcaggtgccggtgctggttcaggtgctggtgctggttcaggagctggtgctggttcaggtgccggttctggttcaggagctggtgctggttcaggtgctggtgctggttcaggagctggagctggatacggagcaggagttggtgctggatacggagtaggatatggagcaggagctggtgctggatacggagcaggagcaggaagcggagctgcctctggtgctggtgccggtgctggagctggtgcaggaacaggctcttctggatttggaccatatgtagcacatggcggatatagcggctacgaatacgcttggtcgtcagaatctgactttggaactggaagcggagctggtgctggttcaggtgctggtgctggttcaggtgctggtgctggctcaggtgctggtgctggttcaggagctggtgctggttcaggtgctggtgctggttcaggagctggagctggatacggagcaggagttggtgctggatacggagcagcatatggagcaggagctggtgctggatacggagctggagcaggaagcggagctgcctctggtgccggtgctggttcaggtgctggtgctggttcaggtgccggtgctggttcaggtgctggtgctggttcaggtgctggtgctggttcaggtgctggtgctggttcaggtgctggtgctggttcaggagctggtgctggttcaggagctggtgctggttcaggtgccggtgctggttcaggagctggagcaggatatggagcaggagctggtgctggatacggagcaggagcaggaagcggagctggctcaggtgctggtgctggttcaggtgctggtgctggttcaggagctggagctggttcaggtgccggtgctggttcaggtgctggtgctggttcaggtgccggttctggttcaggagctggtgctggttcaggtgctggtgctggttcaggagctggagctggatacggagcaggagttggtgctggatacggagcaggatatggagcaggagctggtgctggatacggagcaggagcaggaagcggagctggctcaggtgccggtgctggttcaggagctggagcaggatatggagcaggagctggtgctggatacggagcaggatatggagcaggagctggtgctggatacggagcaggagcaggaaccggagctggctcaggtgctggtgctggttcaggtgctggtgctggttcaggtgctggtgctggttcaggagctggagctggttcaggtgccggtgctggttcaggtgctggtgctggttcaggtgccggttctggttcaggtgctggtgctggttcaggagctggagctggttcaggtgccggtgctggttcaggtgctggtgctggttcaggtgctggtgctggttcaggagctggagctggatacggagcaggagctggtgctggatacggagcaggatatggagcaggagctggtgctggatacggagcaggagcaggaagcggtgctggttcaggtgctggtgctggttcaggtgctggtgctggttcaggtgctggtgctggttcaggagctggtgctggatatggagctggatacggagcaggagctggaagcggagctgcctctggtgctggtgccggtgctggagctggtgcaggaacaggctcttctggatttggaccatatgtagcacatggcggatatagcggctacgaatacgcttggtcgtcagaatctgactttggaactggaagcggagctggtgctggttcaggtgctggtgctggcgcaggtgctggtgctggttcaggagctggagctggatacggagcaggagttggtgctggatacggagcaggatatggagcaggagctggtgctggatacggagcaggagcaggaagcggaactggctcaggtgctggtgctggttcaggagctggagctggatacggagcaggagttggtgctggatacggagcaggagcaggaagcggagctgcctttggtgccggtgctggtgctggtgctggttcaggtgccggtgctggttcaggtgctggtgctggttcaggtgctggtgctggttcaggtgctggtgctggttcaggagctggtgctggatacggagcagggtacggagcaggagttggtgctggatacggagcaggagctggaagcggagctgcctctggtgccggtgctggttcaggtgctggtgctggttcaggtgccggtgctggttcaggtgctggtgctggctcaggtgctggtgctggttcaggagctggagctggatacggagcaggagttggtgctggatacggagcaggatatggagcaggagctggtgctggatacggagctggagcaggaagcggagctgcctctggtgccggtgctggttcaggtgctggtgctggtgctggttcaggtgccggtgctggttcaggtgctggtgctggttcaggtgctggtgctggttcaggtgctggttcaggtgctggtgctggttcaggtgccggtgctggttcaggagctggtgctggatacggagcaggagcaggaagcggagctgcctctggtgctggtgccggtgctggagctggtgcaggaacaggctcttctggatttggaccatatgtagcaaatggcggatatagcggctacgaatacgcttggtcgtcagaatctgactttggaactggaagcggagctggtgctggttcaggtgctggtgctggttcaggtgctggtgctggctcaggtgctggtgctggttcaggagctggagctggatacggagcaggagttggtgctggatacggagcaggatatggagcaggagctggtgctggatacggagcaggagcaggaagcggagctggctcaggtgctggtgctggttcaggagctggagctggttcaggtgccggtgctggttcaggtgctggtgctggttcaggtgccggtgctggttcaggtgctggggctggttcaggagctggtgctggatacggagcaggagcaggaagcggagctgcctctggtgccggtgctggttcaggtgctggtgctggttcaggtgccggtgctggttcaggtgctggtgctggttcaggagctggtgctggttcaggtgctggtgctggttcaggagctggagctggatacggagcaggagttggtgctggatacggagtaggatatggagcaggagctggtgctggatacggagcaggagcaggaagcggagctggctcaggtgctggtgctggttcaggtgctggtgctggttcaggtgccggtgctggttcaggtgctggtgctggttcaggtgccggttcaggtgctggtgctggttcaggagctggtgctggttcaggtgctggtgctggttcaggtgctggttcaggtgctggtgctggctcaggtgctggtgctggatacggagtaggatatggagcaggagctggtgctggatacggagcaggagcaggaagcggagctggctcaggtgctggtgctgggtcaggtgccggtgctggttcaggtgctggtgctggttcaggtgccggttcaggtgctggtgctggttcaggagctggtgctggttcaggtgctggtgctggttcaggagctggagctggatacggagcaggagttggtgctggatacggagtaggatatggagcaggagctggtgctggatacggagcaggagcaggaagcggagctggctcaggtgctggtgctggttcaggtgctggtgctggttcaggtgccggtgctggttcaggtgctggtgctggttcaggagctggtgctggttcaggtgccggtgctggttcaggtgctggtgctggttcaggtgccggttcaggtgctggtgctggttcaggagctggtgctggttcaggtgccggtgctggttcaggtgctggtgctggttcaggtgccggttcaggtgctggtgctggttcaggagctggtgctggttcaggtgctggtgctggttcaggagctggagctggatacggagcaggagttggtgctggatacggagtaggatatggagcaggagttggtgctggatacggagcaggagcaggaagcggagctgcctctggtgccggtgctggttcaggtgctggtgctggtgctggttcaggtgccggtgctggttcaggtgctggtgctggttcaggtgctggtgctggttcaggtgctggtgctggttcaggtgctggtgctggttcaggagctggtgctggatacggagcagggtacggagcaggagttggtgctggatacggagcaggagctggcgttggatacggagcaggagctggcgctggatacggagcaggagctggaagcggagctgcctctggtgccggtgctggtgctggttcaggtgccggtgctggtacaggtgctggggctggttcaggagctggtgctggatacggagcaggagcaggaagcggagctgcctctggtgctggtgccggtgctggagctggtgcaggaacaggctcttctggatttggaccatatgtagcaaatggcggatatagcggctacgaatacgcttggtcgtcagaatctgactttggaactggaagcggagctggtgctggttcaggtgctggtgctggttcaggtgctggtgctggctcaggtgctggtgctggttcaggagctggagctggatacggagcaggagttggtgctggatacggagcaggagcaggaagcggagctggctcaggtgctggtgctggttcaggagctggagctggttcaggtgctggtgctggttcaggagctggtgctggttcaggagctggagctggatacggagcaggagcaggaagcggaactggctcaggtgctggtgctggttcaggtgctggtgctggttcaggtgccggtgctggttcaggtgctggtgctggttcaggagctggtgctggttcaggtgctggtgctggttcaggagttggtgctggatacggagtaggatatggagcaggagctggtgctggatacggagtaggatatggagcaggagctggtgctggatacggagcaggagcaggaagcggaactggctcaggtgctggtgctggttcaggtgctggtgctggttcaggtgccggtgctggttcaggtgctggtgctggttcaggagctggtgctggttcaggtgctggtgctggttcaggagctggagctggatacggagcaggagttggtgctggatacggagtaggatatggagcaggagctggtgctggatacggagcaggagcaggaagcggagctggctcaggtgctggtgctggttcaggtgctggtgctggttcaggtgccggtgctggttcaggtgctggtgctggttcaggtgccggttcaggtgctggtgctggttcaggagctggtgctggttcaggtgctggtgctggttcaggtgctggttcaggtgctggtgctggctcaggtgctggtgctggatacggagtaggatatggagcaggagctggtgctggatacggagcaggagcaggaagcggagctggctcaggtgctggtgctgggtcaggtgccggtgctggttcaggtgctggtgctggttcaggtgccggttcaggtgctggtgctggttcaggagctggtgctggttcaggtgctggtgctggttcaggagctggagctggatacggagcaggagttggtgctggatacggagtaggatatggagcaggagctggtgctggatacggagcaggagcaggaagcggagctggctcaggtgctggtgctggttcaggtgctggtgctggttcaggtgccggtgctggttcaggtgctggtgctggttcaggagctggtgctggttcaggtgccggtgctggttcaggtgctggtgctggttcaggtgccggttcaggtgctggtgctggttcaggagctggtgctggttcaggtgctggtgctggttcaggagctggagctggatacggagcaggagttggtgctggatacggagtaggatatggagcaggagctggtgctggatacggagcaggagcaggaagcggagctgcctctggtgctggtgccggtgctggagctggtgcaggaacaggctcttctggatttggaccatatgtagcaaatggcggatatagcggctacgaatacgcttggtcgtcagaatctgactttggaactggaagcggagctggtgctggttcaggtgctggtgctggttcaggagctggtgctggatacggagcagggtacggagcaggagttggtgctggatacggagcaggagctggcgttggatacggagcaggagctggcgctggatacggagcaggagctggaagcggagctgcctctggtgccggtgctggtgccggtgctggtgctggttcaggtgccggtgctggttcaggtgctggtgctggtgctggttcaggagctggtgctggatacggagcagggtacggaataggagttggtgctggatacggagcaggagctggcgttggatacggagcaggagctggcgctggatacggagcaggagctggaagcggagctgcctctggtgccggtgctggttcaggtgctggtgctggttcaggtgctggtgctggttcaggtgctggtgctggttcaggtgctggtgctggttcaggtgctggtgctggttcaggagctggtgctggatacggagcagggtacggagcaggagttggtgctggatacggagcaggagctggcgttggatacggagcaggagctggcgctggatacggagcaggagctggaagcggagctgcctctggtgccggtgctggtgccggtgctggtgctggtgctggttcaggtgccggtgctggttcaggtgctggtgctggttcaggtgctggtgctggttcaggagctggtgctggttcaggtgctggtgctggttcaggtgctggtgctggttcaggtgctggtgctggctcaggtgctggtgctggttcaggagctggagctggatacggagcaggagttggtgctggatacggagcaggatatggaggagctggtgctggatacggagcaggagcaggaagcggagctgcctctggtgccggtgctggttcaggtgctggtgctggttcaggagctggtgctggttcaggtgctggtgctggttcaggtgctggggctggttcaggtgctggtgctggatacggagcaggagcaggaagcggagctgcctctggtgctggtgccggtgctggagctggtgcaggaacaggctcttctggatttggaccatatgtaaatggcggatatagcggctacgaatacgcttggtcgtcagaatctgactttggaactggaagcggagctggtgctggctcaggtgctggtgctggttcaggagctggagctggatacggagcaggagttggtgctggatacggagcaggatatggagcaggagctggtgctggatacggagcaggagcaggaagcggagctgcctctggtgccggtgctggttcaggtgctggtgctggttcaggtgccggtgctggttcaggtgctggtgctggttcaggtgctggttcaggtgctggtgctggttcaggtgctggtgctggttcaggtgctggtgctggttcaggtgctggtgctggttcaggtgctggtgctggttcaggagctggagctggatacggagcaggagttggtgctggatacggagcaggatatggagcaggagctggtgctggatacggagcaggagcaggaagcggagctgcytctggcgccggtgctggttcaggtgctggtgctggtgctggttcaggtgccggtgctggttcaggtgctggtgctggttcaggtgctggtgctggttcaggtgctggtgctggttcaggtgctggtgctggttcaggtgctggttcaggtgctggtgctggttcaggagctggtgctggatacggagcagggtacggagcaggagttggtgctggatacggagcaggagctggcgttggatacggagcaggagctggcgctggatacggagcaggagctggaagcggagctgcctctggtgccggtgctggttcaggttctggtgctggttcaggtgccggtgctggttcaggtgctggtgctggttcaggtgctggtgctggtgctggttcaggtgctggggctggttcaggagctggtgctggttcaggagctggtgctggatacggagcagggtacggagcaggagcaggaagcggagctgcctctggtgctggtgccggtgctggagctggtgcaggaacaggctcttctggatttggaccatatgtagcaaatggcggatatagcggctacgaatacgcttggtcgtcagaatctgactttggaactggaagcggagctggtgctggctcaggtgctggtgctggttcaggagctggagctggatacggagcaggagttggtgctggttacggagcaggatatggagcaggagctggtgctggatacggagcaggagcaggaagcggagctggctcaggtgccggtgctggttcaggtgctggtgctggttcaggtgccggtgctggttcaggtgctggtgctggttcaggagctggtgctggttcaggtgctggtgctggttcaggtgctggtgctggttcaggtgctggtgctggatacggagcaggatacggagcaggagctggtgctggatacggagcaggagctggcgttggatacggagcaggagctggcgctggatacggagcaggagctggaagcggagctggctctggtgccggtgctggttcaggttctggtgctggtgctggttcaggttctggtgctggttcaggtgctggtgctggttcaggtgctggtgctggttcaggtgctggtgctggttcaggagctggtgctggttcaggtgctggtgctggttcaggtgctggtgctggttcaggtgctggtgctggatacggagcagggtacggaataggagttggtgctggatacggagcaggagctggcgttggatacggagcaggagctggcgctggatacggagcaggagctggaagcggagctgcctctggtgccggtgctggttcaggtgctggtgctggttcaggagctggtgctggttcaggtgctggtgctggttcaggtgctggtgctggttcaggtgctggtgctggttcaggagctggtgctggttcaggtgctggtgctggttcaggtgctggtgctggttcaggagctggtgctggatacggagcaggagctggcgttggatacggagcaggagctggaagcggagctgcctctggtgctggtgctggttcaggtgctggtgctggttcaggtgctggtgctggttcaggtgctggtgctggttcaggtgctggtgctggttcaggtgctggtgctggttcaggtgctggtgctggttcaggtgctggttcaggtgctggtgctggctcaggtgctggtgctggatacggagcagggtacggagcaggagttggtgctggatacggagcaggtgctggatacggagcaggatatggagtaggagctggtgctggatacggagcaggagcaggaagcggagctggctctggtgctggtgctggttcaggtgctggtgctggttcaggtgctggtgctggttcaggtgctggtgctggttcaggtgctggtgctggttcaggtgctggttcaggagctggtgctggatacggagcaggagctggcgctggatacggagcaggagctggcgctggatacggagcaggagctggaagcggagctgcctctggtgctggtgctggtgccggtgctggttcaggtgctggtgctggttcaggtgctggtgctggttcaggtgctggttcaggtgctggtgctggttcaggagctggtgctggatacggagcaggagctggaagcggagctgcctctggtgccggtgctggttcaggtgctggtgctggtgctggtgccggtgctggtgctggttcaggagctggtgctggttcaggagctggtgctggatacggagcaggagcaggaagtggagctgcctctggtgctggtgctggagctggtgcaggaacaggctcttctggatttggaccatatgtagcaaatggcggatatagcagacgtgaaggctacgaatacgcttggtcgtcaaaatctgactttgaaactggaagcggtgctgcctctggtgctggtgctggtgctggttcaggtgctggtgctggttcaggtgccggtgctggttcaggtgctggtgctggttcaggtgccggtgctggtggtagcgtcagttacggagctggcaggggatacggacaaggtgcaggaagtgcagcttcctctgtgtcatctgcttcatctcgcagttacgactattctcgtcgtaacgtccgcaaaaactgtggaattcctagaagacaactagttgttaaattcagagcactgccttgtgtgaattgctaatttttaatataaaataacccttgtttcttacttcgtcctggatacatctatgttttttttttcgttaataaatgagagcatttaagttattgtttttaattacttttttttagaaaacagatttcggattttttgtatgcattttatttgaatgtactaatataatcaattaatcaatgaattcatttatttaagggataacaataatccatgaattcacatgcacatttaaaacaaaactaaattacaataggttcatataaaaacaacaagtatgccttctcaactaagaatactatattgtttaaaccgtaaaaaaagtcatatttctgtatatcaaaacacatctaatattaaaaaaacagtcagcaagcacttacaagtgtgggctcggacagcaattacctggtctcaggagacacttgaagaacgagaagcacgtctctctgtcgattgcgaggctcatgcactatcgcttgagtctgagacctttactgatagggaaatccgtttgagctctcagagggttcggacagcaaaagcttgcccggtctcaggagacattagaagaacgggaagcataactcaataccgatcgcgtttccattgagcctatgcttcgtgataataataaataaagcccaaggtcagacgctaaaagttgtgggtgtagatataagtgttaactgcttttcacacagtcaactcggtcacgcggttatgtagctctgtcacatgctagcaatcctagcaacctgtatgctttgatccccgatggctatacgtctacgtcgtgtatagagaagctt

SEQ ID NO: 2 - DNA, 20 bp, Bombyx mori
gcggcagcacctgcaccagc

SEQ ID NO: 3 - DNA, 20 bp, Bombyx mori
ctgtttgatacgtatggccc

SEQ ID NO: 4 - DNA, 23 bp, Bombyx mori
tatagtattcttagttgagaagg

SEQ ID NO: 5 - DNA, 21 bp, Bombyx mori
agcaacagtaccatcggaaag

SEQ ID NO: 6 - DNA, 21 bp, Bombyx mori
agcactagtgctgcagtcggt

SEQ ID NO: 7 - DNA, 21 bp, Bombyx mori
aatgtgcatgtgaattcatgg

SEQ ID NO: 8 - DNA, 21 bp, Bombyx mori
ttacttcgtcctggatacatc

SEQ ID NO: 9 - DNA, 7309 bp, Unknown
  repeating motifs of MaSp2 and flagelliform
ggcggtgcaggtccaggcggtgcgggtccaggcggagcaggcccaggcggagcaggtccaggcggagcgggtccaggtggtgctggccccggtggagccggtcctggcggtgctggacctggaggcccatcaggtcctggttctgctgccgcagctgctgccgctgccggcccaggtggagccggtccgggcggtgctggacccggaggcgccggccctggtggagctggtccaggcggtgccggaccgggaggcgctggccccggtggagccggtcctggcggtgctggacccggaggccctagcggaccaggctccgctgctgcagccgcagctgcagctggcccaggtggagccggtccgggcggtgctggacccggaggcgccggccctggtggagctggtccaggcggtgccggaccgggaggcgctggccccggtggagccggtcctggcggtgctggaccgggaggcccctcgggtcctggaagtgctgccgcagcagcagctgcagcaggcccaggtggagccggtccgggcggtgctggacccggaggcgccggccctggtggagctggtccaggcggtgccggaccgggaggcgctggccccggtggagccggtcctggcggtgctggtcctggtggtccttcaggccccggttctgctgccgcagcagccgctgcagcgggcccaggtggagccggtccgggcggtgctggacccggaggcgccggccctggtggagctggtccaggcggtgccggaccgggaggcgctggccccggtggagccggtcctggcggtgctggacctggaggccccagcggaccgggctctgcagctgccgctgctgctgctgctggcccaggtggagccggtccgggcggtgctggacccggaggcgccggccctggtggagctggtccaggcggtgccggaccgggaggcgctggccccggtggagccggtcctggcggtgctggacccggaggcccctcgggtccaggaagtgctgccgcagcggcagctgcagccggcccaggtggagccggtccgggcggtgctggacccggaggcgccggccctggtggagctggtccaggcggtgccggaccgggaggcgctggccccggtggagccggtcctggcggtgctggaccgggaggcccttcaggccctggttctgctgccgccgctgcagctgccgcaggcccaggtggagccggtccgggcggtgctggacccggaggcgccggccctggtggagctggtccaggcggtgccggaccgggaggcgctggccccggtggagccggtcctggcggtgctggtcctggtggtccgagcggacccggctctgcagctgcggctgccgctgcagctggcccaggtggagccggtccgggcggtgctggacccggaggcgccggccctggtggagctggtccaggcggtgccggaccgggaggcgctggccccggtggagccggtcctggcggtgctggacctggaggcccttctggtccgggaagtgctgccgcagccgcagctgccgccggcccaggtggagccggtccgggcggtgctggacccggaggcgccggccctggtggagctggtccaggcggtgccggaccgggaggcgctggccccggtggagccggtcctggcggtgctggacccggaggcccatcaggcccaggttctgctgccgcagcggccgctgccgccggcccaggtggagccggtccgggcggtgctggacccggaggcgccggccctggtggagctggtccaggcggtgccggaccgggaggcgctggccccggtggagccggtcctggcggtgctggaccgggaggcccaagcggacctggctccgctgctgccgccgcagctgcggctggcccaggtggagccggtccgggcggtgctggacccggaggcgccggccctggtggagctggtccaggcggtgccggaccgggaggcgctggccccggtggagccggtcctggcggtgctggtcctggtggtccttctggtcccggaagtgctgccgccgctgccgctgcggcaggcccaggtggagccggtccgggcggtgctggacccggaggcgccggccctggtggagctggtccaggcggtgccggaccgggaggcgctggccccggtggagccggtcctggcggtgctggacctggaggcccgtctggtcctggttcagctgccgcagcagcagcagcggcgggcccaggtggagccggtccgggcggtgctggacccggaggcgccggccctggtggagctggtccaggcggtgccggaccgggaggcgctggccccggtggagccggtcctggcggtgctggacccggaggcccgtctggaccaggctctgctgccgcagcagcggctgcggccggcccaggtggagccggtccgggcggtgctggacccggaggcgccggccctggtggagctggtccaggcggtgccggaccgggaggcgctggccccggtggagccggtcctggcggtgctggaccgggaggcccgtcaggtcctggatctgctgccgcagcagctgctgcagccggcccaggtggagccggtccgggcggtgctggacccggaggcgccggccctggtggagctggtccaggcggtgccggaccgggaggcgctggccccggtggagccggtcctggcggtgctggtcctggtggtcctagcggccccggttcagcagccgctgccgcagcagctgcgggcccaggtggagccggtccgggcggtgctggacccggaggcgccggccctggtggagctggtccaggcggtgccggaccgggaggcgctggccccggtggagccggtcctggcggtgctggacctggaggcccctctggaccgggctctgcggctgcagctgctgctgcagctggcccaggtggagccggtccgggcggtgctggacccggaggcgccggccctggtggagctggtccaggcggtgccggaccgggaggcgctggccccggtggagccggtcctggcggtgctggacccggaggccccagcggtccaggatctgcagccgctgccgcagcggcggcgggcccaggtggagccggtccgggcggtgctggacccggaggcgccggccctggtggagctggtccaggcggtgccggaccgggaggcgctggccccggtggagccggtcctggcggtgctggaccgggaggccccagcggccctggttcagcagctgcagccgcagctgctgcaggcccaggtggagccggtccgggcggtgctggacccggaggcgccggccctggtggagctggtccaggcggtgccggaccgggaggcgctggccccggtggagccggtcctggcggtgctggtcctggtggtccaagcggacccggctctgcggctgccgctgccgctgcagctggcccaggtggagccggtccgggcggtgctggacccggaggcgccggccctggtggagctggtccaggcggtgccggaccgggaggcgctggccccggtggagccggtcctggcggtgctggacctggaggcccctctggtccgggatctgcagctgcagccgccgctgctgccggcccaggtggagccggtccgggcggtgctggacccggaggcgccggccctggtggagctggtccaggcggtgccggaccgggaggcgctggccccggtggagccggtcctggcggtgctggacccggaggccccagtggcccaggttcagcagctgccgctgcagctgctgccggcccaggtggagccggtccgggcggtgctggacccggaggcgccggccctggtggagctggtccaggcggtgccggaccgggaggcgctggccccggtggagccggtcctggcggtgctggaccgggaggccccagtggacctggctccgctgccgcagccgcagcagcagccggcccaggtggagccggtccgggcggtgctggacccggaggcgccggccctggtggagctggtccaggcggtgccggaccgggaggcgctggccccggtggagccggtcctggcggtgctggtcctggtggtccttctggtcccggatctgctgccgcagcggcggctgctgcgggcccaggtggagccggtccgggcggtgctggacccggaggcgccggccctggtggagctggtccaggcggtgccggaccgggaggcgctggccccggtggagccggtcctggcggtgctggacctggaggccccagtggtcctggttccgcagccgctgccgcagccgccgccggcccaggtggagccggtccgggcggtgctggacccggaggcgccggccctggtggagctggtccaggcggtgccggaccgggaggcgctggccccggtggagccggtcctggcggtgctggacccggaggccccagcggaccaggctctgcagccgctgccgccgctgctgccggcccaggtggagccggtccgggcggtgctggacccggaggcgccggccctggtggagctggtccaggcggtgccggaccgggaggcgctggccccggtggagccggtcctggcggtgctggaccgggaggccccagcggtcctggatcagcagccgctgccgccgcggcagcgggcccaggtggagccggtccgggcggtgctggacccggaggcgccggccctggtggagctggtccaggcggtgccggaccgggaggcgctggccccggtggagccggtcctggcggtgctggtcctggtggtcctagtggccccggttcagccgctgcagccgcagcagccgcgggcccaggtggagccggtccgggcggtgctggacccggaggcgccggccctggtggagctggtccaggcggtgccggaccgggaggcgctggccccggtggagccggtcctggcggtgctggacctggaggccccagcggaccgggctctgctgctgccgctgcagctgctgctggcccaggtggagccggtccgggcggtgctggacccggaggcgccggccctggtggagctggtccaggcggtgccggaccgggaggcgctggccccggtggagccggtcctggcggtgctggacccggaggccccagcggtccaggatctgctgctgcagctgcagctgcagctggcccaggtggagccggaccgggcggtgctggtcccggaggcgccggacctggtggagctggtccaggcggtgccggaccgggaggcgctggtcccggtggagccggacctggcggtgctggtccgggaggcccctctggccctggttcagctgccgcggctgctgccgctgccggaccaggtggagccggtccgggcggtgctggacccggaggcgccggtcctggtggagctggaccaggcggtgccggtccgggaggcgctggacccggtggagccggtcctggcggtgctggtcctggtggtccttctggacccggctctgctgctgctgctgctgccgctgccggtccaggtggagctggaccgggcggtgctggtcccggaggcgctggacctggtggagctggtccaggcggtgctggaccgggaggcgctggtcccggtggagctggacctggcggtgctggtcctggaggcccatctggtccgggatctgcggctgcagctgctgctgccgctggaccaggtggagctggtccgggcggtgctggacccggaggcgctggtcctggtggagctggaccaggcggtgctggtccgggaggcgctggacccggtggagctggtcctggcggtgctggacccggaggccccagcggcccaggttcagcggctgccgctgcggctgcagctggtccaggtggagctggaccgggcggtgctggtcccggaggcgctggacctggtggagctggtccaggcggtgctggaccgggaggcgctggtcccggtggagctggacctggcggtgctggtccgggaggccctagcggacctggctctgcagctgcagctgccgctgccgcaggaccaggtggagctggtccgggcggtgctggacccggaggcgctggtcctggtggagctggaccaggcggtgctggtccgggaggcgctggacccggtggagctggtcctggcggtgctggtcctggtggtccttctggtcccggatctgcagctgcagctgcggctgctgctggtccaggtggagctggaccgggcggtgctggtcccggaggcgctggacctggtggagctggtccaggcggtgctggaccgggaggcgctggtcctggtggagctggaccaggcggtgctggtcctggaggccctagtggtcctggttctgcagctgcagctgctgctgctgctggaccgggtggagctggtcctggcggtgctggtcctggtggtgctggtccgggtggagctggacctggcggtgctggtcctggtggtgctggaccgggtggagctggtcctggcggtgctggacccggaggccccagcggaccaggctctgcggctgcggctgctgctgccgcaggtccaggtggagctggaccgggcggtgctggtcctggaggcgctggaccaggtggagctggtccgggcggtgctggacctggaggcgctggtccaggtggagctggaccgggcggtgctggtccgggaggcccatctggtcctggatctgcagctgcggctgctgctgctgctggacctggtggagctggtccaggcggtgctggaccgggaggcgctggtcctggtggagctggaccaggcggtgctggtccgggaggcgctggacctggtggagctggtccaggcggtgctggtcctggtggtcctagcggccccggtagcgctgctgccgctgccgctgccgctggtccgggtggagctggacctggcggtgctggtcctggtggtgctggaccgggtggagctggtccaggcggtgctggacctggaggcgctggtccaggtggagctggacctggcggtgctggtcctggaggcccgtctggaccgggctcagcagctgcagctgccgcagcagccggaccaggtggagctggtcctggcggtgctggtcctggtggtgctggtcctggtggagctggtccaggcggtgctggtcctggaggcgctggtcctggtggagctggtcctggaggagcaggtccaggtggtccctcaggtccaggttcggcggcggcggcggcagcagcagcggatatcg

SEQ ID NO: 10 - DNA, 975 bp, Unknown
  repeating motifs of MaSp2 and flagelliform
cccccaggtggagctgggcccggtggagcggggcctggaggagcaggtccaggcggcgcaggaccgggaggtgcgggccctggcggtgctggacctggcggggcagggcccggaggcgcgggacctggaggtccgagcgggcctggctcggcagcggctgcagcagctgcggcagggcccggtggagcggggcctggaggagcaggtccaggcggcgcaggaccgggaggtgcgggccctggcggtgctggacctggcggggcagggcccggaggcgcgggacctggaggtccgagcgggcctggctcggcagcggctgcagcagctgcggcagggcccggtggagcggggcctggaggagcaggtccaggcggcgcaggaccgggaggtgcgggccctggcggtgctggacctggcggggcagggcccggaggcgcgggacctggaggtccgagcgggcctggctcggcagcggctgcagcagctgcggcagggcccggtggagcggggcctggaggagcaggtccaggcggcgcaggaccgggaggtgcgggccctggcggtgctggacctggcggggcagggcccggaggcgcgggacctggaggtccgagcgggcctggctcggcagcggctgcagcagctgcggcagggcccggtggagcggggcctggaggagcaggtccaggcggcgcaggaccgggaggtgcgggccctggcggtgctggacctggcggggcagggcccggaggcgcgggacctggaggtccgagcgggcctggctcggcagcggctgcagcagctgcggcagggcccggtggagcggggcctggaggagcaggtccaggcggcgcaggaccgggaggtgcgggccctggcggtgctggacctggcggggcagggcccggaggcgcgggacctggaggtccgagcgggcctggctcggcagcggctgcagcagctgcggcatccggg

SEQ ID NO: 11 - DNA, 3165 bp, Caerostris darwini
atggccggcggcgctggcgggcgcgggggtctaggcgggcagggaggcggacagggagctggcggcgcagggcaaggggggtacgggtccggcctaggcgggctaggaggcggagctgcagcggcagctgctgctgcagggggagcaggcggtttgggcggacaaggagggggccaaggtgcaggccaaggaggatacggttctgggcagggggggcagggggctggttccgcagcggcagcggcagcagccggaggagccggtgggaggggcggtctgggggggcaaggtggggggcagggagcgggaggggcaggtcaaggcggttatgggtcaggcttaggtggtctgggtggtggggcggcggctgccgccgcagcagctggtggggctggcggcctgggtggtcaagggggcggtcaaggcgcgggtcagggtggctatggctcgggtcaaggcgggcaaggcgcgggttccgcggcagccgcagctgcggccggcggccgtggtgggtacggcggtcagggtggtggtcagggtgctggtggagccgggcagggtgggtatggctccggtctcggcggcctagggggcggtgccgcagctgctgcagcggcggcaggcggagcgggcggcctcggagggcaagggggaggacagggtgcgggccagggcggctatgggtccgggcaaggagggcaaggagctggtagtgcagcggccgcagcagctgctggcggggctggcggtctgggcggttacggtgggcaaggagggggtcaaggcgctggaggcgctgggcaaggggggtacgggtcgggcctcggcggattaggcggtggggcggctgccgcggcagctgccgcgggcggggcgggtggacttgtaggacaaggagggggccagggagcggggcaagggggctatggcagcggccaggggggtcaaggagcaggcagtgcggcagccgctgctgccgcgggaggcgcaggtggtcgcggcggactcggaggacaaggcgggggacaaggagcgggtggtgctggacaaggaggctacggcagtggtctgggtgggctcggaggaggggcctccgccgccgcggcagcggcgggtggagctgggggcctgggtggtcagggaggcggtcaaggtgccgggcaggggggttacggatccgggcaaggcggccaaggggcaggctcagcagccgcagccgccgctgcaggaggttctgggggtctgggcggtcagggagggtatgggggtcaaggcggatatggagggggatacggtggtcagcaacaaggtggcggccagggtgcaggtcaaggtggttatggatcgggccaagggggccaaggtgcgggttcagcggctgcagcggccgctgccggtgggcgcggcggttatggcggtcagggggggggccagggtgccggcggagcaggtcaaggtggatacggctccgggcttgggggattgggtggtggggcggctgccgccgcaggcggtgcgggcggtctgggtggtcaaggtggcggacaaggtgccggtcaaggcgggtatggatcagggcagggcggacagggcgcggggtcagctgccgcggctgcggccgcaggagggcgcggtggatatggtgggcaaggtggcggccagggagccgggggagccggacagggtggctacggctccggcctaggcggtttgggtggcggggcagccgcggccgctgcggctgcagggggtgcagggggtctggcgggccagggcgggggccaaggagcaggacagggggggtatggctcggggcagggtggccaaggggcaggatctgccgctgccgctgccgcggcaggtggagctggtggccgtggaggttatggtgggcaaggcggtcagggagctggtggagcggggcaaggtgggtatggttccggattgggtggattgggcggtggcgccgcagccgctgccgcagctgctgggggtgctggggggttagggggtcaaggtggtgggcagggagccggtcagggtggttatggcagtggccagggaggccaaggcgcaggtagtgctgcagccgctgcagcggccggcggggcagggggtcaaggcggatacggtggacaaggcggacaaggagcaggtggcgccggacaaggtggttatggttcagggctgggaggagtcggtggtggcgccgccgcaggcggagccggtggactcggtggtcagggcggcggacagggtgcgggccagggtggctacggatcagggcagggtggccagggcgctggatcggctgctgcggccggtcgaggtgggtatggtggacgtgggggtgcaggctctagtagcgctgccgcagcggccggatcgggcggtgatggctccggttctggcggctacggggggaggggcgggcaaggtggagacggcgcttcctccgctgcagcggcagctggaagcggtggcgatggctcagggtctgggggctacggaggtaggggaggccgcggaggtcaaggtggagcaggatcgtcgtcagcagcagctgcagccgggtcagggggagacgggtctggaagtggtggatatgggggacgagggggtcgtggaggcgcagggtcctcatccgccgccagtgctgccgccgggggggaggacgggtttgggcgcggaggttatggagggagaggtggagccggctccagctcagcggctgcggctgcgggctctggtggcgacggtagcggcagcgggggttacggtggtcgcggtgggcaaggcggagccggttcatcgagtgctgcggcggctggttcggggggtgatggttatggaagtgggggctatggtggcagaggaggacaaggtggagctgggtcgtcatcggccgcctcggccgcaggttccgggggcgatggtttcgggtccggtttctatgggggcagaggcggagagggtggagcaggcagctccagtgctgctgcagccgccggctcaggtggagatggatacggctctggtggttacggtggtagaggaggtcaaggtggtgctgggggcgcgagtgcctcggccgtcgccgcggggggagggcgtggtcagggcgggtatggcggcagggggggacaaggcggagccggaactaga

SEQ ID NO: 12 - DNA, 6325 bp, Caerostris darwini
atggccggcggcgctggcgggcgcgggggtctaggcgggcagggaggcggacagggagctggcggcgcagggcaaggggggtacgggtccggcctaggcgggctaggaggcggagctgcagcggcagctgctgctgcagggggagcaggcggtttgggcggacaaggagggggccaaggtgcaggccaaggaggatacggttctgggcagggggggcagggggctggttccgcagcggcagcggcagcagccggaggagccggtgggaggggcggtctgggggggcaaggtggggggcagggagcgggaggggcaggtcaaggcggttatgggtcaggcttaggtggtctgggtggtggggcggcggctgccgccgcagcagctggtggggctggcggcctgggtggtcaagggggcggtcaaggcgcgggtcagggtggctatggctcgggtcaaggcgggcaaggcgcgggttccgcggcagccgcagctgcggccggcggccgtggtgggtacggcggtcagggtggtggtcagggtgctggtggagccgggcagggtgggtatggctccggtctcggcggcctagggggcggtgccgcagctgctgcagcggcggcaggcggagcgggcggcctcggagggcaagggggaggacagggtgcgggccagggcggctatgggtccgggcaaggagggcaaggagctggtagtgcagcggccgcagcagctgctggcggggctggcggtctgggcggttacggtgggcaaggagggggtcaaggcgctggaggcgctgggcaaggggggtacgggtcgggcctcggcggattaggcggtggggcggctgccgcggcagctgccgcgggcggggcgggtggacttgtaggacaaggagggggccagggagcggggcaagggggctatggcagcggccaggggggtcaaggagcaggcagtgcggcagccgctgctgccgcgggaggcgcaggtggtcgcggcggactcggaggacaaggcgggggacaaggagcgggtggtgctggacaaggaggctacggcagtggtctgggtgggctcggaggaggggcctccgccgccgcggcagcggcgggtggagctgggggcctgggtggtcagggaggcggtcaaggtgccgggcaggggggttacggatccgggcaaggcggccaaggggcaggctcagcagccgcagccgccgctgcaggaggttctgggggtctgggcggtcagggagggtatgggggtcaaggcggatatggagggggatacggtggtcagcaacaaggtggcggccagggtgcaggtcaaggtggttatggatcgggccaagggggccaaggtgcgggttcagcggctgcagcggccgctgccggtgggcgcggcggttatggcggtcagggggggggccagggtgccggcggagcaggtcaaggtggatacggctccgggcttgggggattgggtggtggggcggctgccgccgcaggcggtgcgggcggtctgggtggtcaaggtggcggacaaggtgccggtcaaggcgggtatggatcagggcagggcggacagggcgcggggtcagctgccgcggctgcggccgcaggagggcgcggtggatatggtgggcaaggtggcggccagggagccgggggagccggacagggtggctacggctccggcctaggcggtttgggtggcggggcagccgcggccgctgcggctgcagggggtgcagggggtctggcgggccagggcgggggccaaggagcaggacagggggggtatggctcggggcagggtggccaaggggcaggatctgccgctgccgctgccgcggcaggtggagctggtggccgtggaggttatggtgggcaaggcggtcagggagctggtggagcggggcaaggtgggtatggttccggattgggtggattgggcggtggcgccgcagccgctgccgcagctgctgggggtgctggggggttagggggtcaaggtggtgggcagggagccggtcagggtggttatggcagtggccagggaggccaaggcgcaggtagtgctgcagccgctgcagcggccggcggggcagggggtcaaggcggatacggtggacaaggcggacaaggagcaggtggcgccggacaaggtggttatggttcagggctgggaggagtcggtggtggcgccgccgcaggcggagccggtggactcggtggtcagggcggcggacagggtgcgggccagggtggctacggatcagggcagggtggccagggcgctggatcggctgctgcggccggtcgaggtgggtatggtggacgtgggggtgcaggctctagtagcgctgccgcagcggccggatcgggcggtgatggctccggttctggcggctacggggggaggggcgggcaaggtggagacggcgcttcctccgctgcagcggcagctggaagcggtggcgatggctcagggtctgggggctacggaggtaggggaggccgcggaggtcaaggtggagcaggatcgtcgtcagcagcagctgcagccgggtcagggggagacgggtctggaagtggtggatatgggggacgagggggtcgtggaggcgcagggtcctcatccgccgccagtgctgccgccgggggggaggacgggtttgggcgcggaggttatggagggagaggtggagccggctccagctcagcggctgcggctgcgggctctggtggcgacggtagcggcagcgggggttacggtggtcgcggtgggcaaggcggagccggttcatcgagtgctgcggcggctggttcggggggtgatggttatggaagtgggggctatggtggcagaggaggacaaggtggagctgggtcgtcatcggccgcctcggccgcaggttccgggggcgatggtttcgggtccggtttctatgggggcagaggcggagagggtggagcaggcagctccagtgctgctgcagccgccggctcaggtggagatggatacggctctggtggttacggtggtagaggaggtcaaggtggtgctgggggcgcgagtgcctcggccgtcgccgcggggggagggcgtggtcagggcgggtatggcggcagggggggacaaggcggagccggaatggctggtggagccggcggtagaggtggtttgggtggacagggcggtggacaaggagctggcggtgccggtcaaggtggttacggatctggtctgggtggattgggcggtggagctgcagcagctgctgctgctgccggcggtgctggaggcctcggtggacagggcggtggacagggtgctggacagggcggttacggatctggtcagggaggccaaggagctggatctgcagctgcagctgctgctgctggtggagccggcggtagaggtggtctgggtggacaaggcggtggacagggcgctggcggtgccggacaaggtggttacggttctggactcggtggactgggcggtggagccgctgctgctgctgctgccgctggcggtgctggaggcttgggtggacaaggcggtggacaaggtgctggtcaaggcggttacggaagtggccagggaggccaaggtgctggatcagctgccgctgcagctgctgccggtggaagaggcggttacggaggccagggtggaggccagggagctggtggagccggtcagggcggttatggttctggtttgggaggcctcggtggaggcgctgctgcagctgcagccgcagccggtggagctggcggtctgggaggccagggtggaggccaaggagctggccagggtggatacggttctggacagggcggtcaaggcgctggttctgcggctgcagctgccgctgctggaggcgccggtggattgggcggttacggaggccaaggtggaggccaaggtgctggtggagccggacaaggcggttatggatctggcctgggaggcttgggtggaggcgccgcagctgcagctgctgccgctggtggagctggcggtctcgtgggacaaggtggtggtcagggagctggacaaggtggttacggctccggtcagggtggacaaggagctggttctgctgcggctgcagctgctgccggcggtgccggaggcagaggtggactgggcggtcagggaggcggtcaaggagctggaggcgccggtcagggtggatacggtagtggactcggcggtctgggaggcggtgcttctgcggctgccgctgctgccggaggcgccggtggactcggcggtcagggaggcggtcagggtgctggtcagggaggctacggaagtggccagggtggacaaggcgcaggatcagctgctgccgctgccgctgctggcggtagcggaggcctgggtggacaaggcggttacggaggccagggtggatacggcggtggatacggcggtcaacagcaaggtggtggtcaaggcgcaggacagggaggctacggatcaggtcagggtggacaaggcgccggttctgctgcggctgcggctgctgccggcggtagaggtggttacggtggacaaggcggtggacagggggctggcggtgccggccaaggtggttatggttctggattgggtggactcggcggtggagctgctgctgctgctggcggtgctggaggcttgggtggacaaggcggtggacagggagctggacaaggcggttacggttctggacagggaggccagggtgctggttctgctgccgctgcggcagcagccggtggaagaggcggttatggaggccagggtggaggccagggggctggtggagccggacagggcggttatggttctgggctgggaggcttgggtggaggcgctgctgccgctgcagcagcagccggtggagctggcggtttggctggacagggaggcggtcagggagctggacagggaggctacggcagtggacagggtggacagggagctggttctgcggctgctgctgcagctgctggcggtgctggaggcagaggtggatacggcggtcagggaggccagggtgctggtggagccggtcaaggcggttacggttccggactcggaggcctgggtggaggcgccgctgcagcagccgcagctgctggtggagctggcggtctgggaggccaaggtggaggccagggtgcaggacaaggtggatacggatctggtcagggcggtcaaggtgctggatctgcggcagctgccgctgcagccggaggcgctggtggacaaggcggttacggaggccagggtggacagggcgctggcggtgccgggcagggaggctatggttcaggtttgggtggagtgggcggtggagctgctgctggcggtgctggaggcctcggtggacagggcggtggacaaggggcaggtcagggcggttacggctctggtcagggaggccagggtgccggttctgctgctgctgctggaagaggtggatatggcggtagaggtggtgctggatcatcttctgctgctgctgctgctggttccggtggagatggaagtggctcaggcggttacggaggcagaggtggacaaggcggtgacggagcttcctctgcggctgcagctgcgggatcgggaggcgatggttctggaagcggtggatacggcggtagaggtggtagaggtggacagggcggtgctggaagttcatcagctgccgctgccgctggttctggaggcgacggatctggttcgggtggatacggcggtagaggtggtagaggtggagctggttcttcttctgctgcttctgccgctgctggcggtgaagacggattcggcagaggtggttatggtggaagaggcggtgctggttcttcatcagctgcggctgcagctggtagtggaggcgatggtagtggatcaggtggatacggcggtagaggtggtcaaggtggagctggatcttcttctgcagctgcagctggtagtggcggtgacggttacggatcaggtggttacggtggaagaggcggtcagggaggcgctggttcatcatcagctgcttctgctgctggttcgggtggagatggtttcggatctggtttctacggcggtagaggtggtgaaggtggtgctggaagctcttctgcagctgccgcagcaggttctggcggtgacggatacggtagcggaggctacggtggaagaggcggtcaaggtggtgctggtggtgcttctgcttctgctgttgctgctggcggtggaagaggccaaggcggttatggaggcagaggtggacagggcggtgctggggatatcg

SEQ ID NO: 13 - DNA, 1293 bp, Nephila clavipes
ctaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatgggggatccgggctaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatggggga

SEQ ID NO: 14 - DNA, 2907 bp, Caerostris darwini
atgccccaatcaccttacggacccggtccccagggacctggaccacaaggtcccgggccccaggggccgtccggacctggaccgcaacgtccgcagggcccagggccgcaggggccttacggccccggcggtgtttccgttgtctcagcgacagtatccggtccggggcctcaggggcctagtggccccggcccacaggggccctacgggccaggtccgcagggtcctggtccgcaagggcccggcccacaattgccgggaccacagggtccatctgggcctggaccccaaggtccgtatggcccaggaccacaaggtccaggccctcagggcccgggccctcaaggaccatccggacccggtccgcaacaaccacaaggtccgggtcctcaaagaccctatggaccaggaggggtatctgttgtatctacaactgtatccgggcccggtccgcagggccccctcgggccaggggctcaagtgccgtacggtccgggcccgcaagtacccgggccacaaggaccaggaccacagggtccaagcggtccgggtccacaacgcccccaaggacctggaccccagggtccgtacggtccgggcggtgtttctgtggtgtcccaaactgttagtggcccagggccgcaggggccctccggccctggaccgcagggcccgtatggacccggaccccaaggaccaggacctcaaggtccggcgccccaaggtccgtctggcccgggacctcagcgcccgcagggaccaggaccgcaacgtccatatggtccaggtggcatatcagtagtttccactaccgttagcgggcctggacctcaggggccttctgctcccggtcctcaaggtccctacggccccggccctcaagtaccggggccccaaggcccggggccccaaggcccatcaggaccagggccacagaggccacaagggccaggtccgcaaggtccttacggcccggggggtgtttctgttgtttcccagacagtttctggaccgggaccgcaaggcccgtctggtccaggtccccaggggccttatggtcccggaccgcagggtcccgggcctcagggacccggccctcaaggaccctccggagcgggccctcaacgtccacaaggccctgggccacagggtccttatggtccggggggcgtctcagtcgtctccgctacagtaagcggtccaggcccccaaggaccatcaggtccagggccacaaggcccgtatgggccaggaccgcaggggccaggcccacagcgccctgtgccgcaaggaccgagcggacccagaccacaacagccccaaggtccagggccacaaaggccttttggaccggggggagtctcagcggtgagcaccactgtgttcggacctggaccccagggtccctcgggcccagggccgcagggcccttatgggccgggccctcaaggtccgggcccacagggacccggtccgcagggaccgtcaggccaaggaccgcaacgtccaagtgggccacgaccgcaaggaccctacggccccggcggtatatcagttgtatccgcgacggtaagcgggcccggccctcaagggccctcaggaccaggtccccaacgcccatatggccccgggcctgagggtccgggcccgcaaggagcgggccctcaaggcccaggactacagcgtccgtcgggacctgggccacagggcccttacggtcctggtccgagaggtcccccgcagggtccctacggtccggggccgcaagggccaggacctcaaggccctggtcctcaagggcccgggccccgaggaccctccgggccaggaccacagggcccgtacggaccgggtggcgtatcagttgtgtcggcctcggtttcgggacccggccctcaggggccctctggtcccgggccacaagggccttatggcccggggccgcaaggaccgggtccacaaggcccgggccctcagggcccgggcccgcaggggcctggtcccaggggtcctagcggtccagggcctcaaggtccttatggtcccggcggtgtgagcgtcgcctctgcttcagtgtccggaccgggaccacagggtccatccggtcccgggcctcaagggccttatggaccgggccctcagggcccgggtccgcgtggaccggggccgcaggggcctggtccccagggacccggacctcagggacctagtggccctgggccgcagggcccgtatgggcctgggggggtatccgtagtgagtgcgtcggtttcaggtcccggtcctcagggtccgtctggcccaggaccgcagggaccatacggccccggtccccaaggtcctggaccccagggtcctggtcctcagggcccgggcccgcaagggccggggccgagaggaccttcgggcccaggtcctcaagggccttatggcccgggtggtgtcagtgtggtatctgcaagtgttagcggccccggtccgcaaggcccttctggacccggcccccaaggcccgtacgggccgggaccccagggtcctggcccgcaagtgccaggtccgcagggacccggtccccaaggtccagggccacaagggccttcgggccctggacctcagggcccttatggcccaggcggcgtaagcgtggtatccgcgagtgtatcaggtcctggtccccaaggtccgagcggtcccgggccccaagggccatacgggccgggcccacaggggccaggcccacgtggccctggtccccaagggccaggaccgcaaggtccaggcccacagggaccgagcggcccgggcccgcaggggccgtacggaccaggcggagtcagcgttgttagcgccagcgtgtccgggccagggccacaagggccgtctggtcccactagtgctgcagtcggt

SEQ ID NO: 15 - DNA, 5785 bp, Caerostris darwini
atgccccaatcaccttacggacccggtccccagggacctggaccacaaggtcccgggccccaggggccgtccggacctggaccgcaacgtccgcagggcccagggccgcaggggccttacggccccggcggtgtttccgttgtctcagcgacagtatccggtccggggcctcaggggcctagtggccccggcccacaggggccctacgggccaggtccgcagggtcctggtccgcaagggcccggcccacaattgccgggaccacagggtccatctgggcctggaccccaaggtccgtatggcccaggaccacaaggtccaggccctcagggcccgggccctcaaggaccatccggacccggtccgcaacaaccacaaggtccgggtcctcaaagaccctatggaccaggaggggtatctgttgtatctacaactgtatccgggcccggtccgcagggccccctcgggccaggggctcaagtgccgtacggtccgggcccgcaagtacccgggccacaaggaccaggaccacagggtccaagcggtccgggtccacaacgcccccaaggacctggaccccagggtccgtacggtccgggcggtgtttctgtggtgtcccaaactgttagtggcccagggccgcaggggccctccggccctggaccgcagggcccgtatggacccggaccccaaggaccaggacctcaaggtccggcgccccaaggtccgtctggcccgggacctcagcgcccgcagggaccaggaccgcaacgtccatatggtccaggtggcatatcagtagtttccactaccgttagcgggcctggacctcaggggccttctgctcccggtcctcaaggtccctacggccccggccctcaagtaccggggccccaaggcccggggccccaaggcccatcaggaccagggccacagaggccacaagggccaggtccgcaaggtccttacggcccggggggtgtttctgttgtttcccagacagtttctggaccgggaccgcaaggcccgtctggtccaggtccccaggggccttatggtcccggaccgcagggtcccgggcctcagggacccggccctcaaggaccctccggagcgggccctcaacgtccacaaggccctgggccacagggtccttatggtccggggggcgtctcagtcgtctccgctacagtaagcggtccaggcccccaaggaccatcaggtccagggccacaaggcccgtatgggccaggaccgcaggggccaggcccacagcgccctgtgccgcaaggaccgagcggacccagaccacaacagccccaaggtccagggccacaaaggccttttggaccggggggagtctcagcggtgagcaccactgtgttcggacctggaccccagggtccctcgggcccagggccgcagggcccttatgggccgggccctcaaggtccgggcccacagggacccggtccgcagggaccgtcaggccaaggaccgcaacgtccaagtgggccacgaccgcaaggaccctacggccccggcggtatatcagttgtatccgcgacggtaagcgggcccggccctcaagggccctcaggaccaggtccccaacgcccatatggccccgggcctgagggtccgggcccgcaaggagcgggccctcaaggcccaggactacagcgtccgtcgggacctgggccacagggcccttacggtcctggtccgagaggtcccccgcagggtccctacggtccggggccgcaagggccaggacctcaaggccctggtcctcaagggcccgggccccgaggaccctccgggccaggaccacagggcccgtacggaccgggtggcgtatcagttgtgtcggcctcggtttcgggacccggccctcaggggccctctggtcccgggccacaagggccttatggcccggggccgcaaggaccgggtccacaaggcccgggccctcagggcccgggcccgcaggggcctggtcccaggggtcctagcggtccagggcctcaaggtccttatggtcccggcggtgtgagcgtcgcctctgcttcagtgtccggaccgggaccacagggtccatccggtcccgggcctcaagggccttatggaccgggccctcagggcccgggtccgcgtggaccggggccgcaggggcctggtccccagggacccggacctcagggacctagtggccctgggccgcagggcccgtatgggcctgggggggtatccgtagtgagtgcgtcggtttcaggtcccggtcctcagggtccgtctggcccaggaccgcagggaccatacggccccggtccccaaggtcctggaccccagggtcctggtcctcagggcccgggcccgcaagggccggggccgagaggaccttcgggcccaggtcctcaagggccttatggcccgggtggtgtcagtgtggtatctgcaagtgttagcggccccggtccgcaaggcccttctggacccggcccccaaggcccgtacgggccgggaccccagggtcctggcccgcaagtgccaggtccgcagggacccggtccccaaggtccagggccacaagggccttcgggccctggacctcagggcccttatggcccaggcggcgtaagcgtggtatccgcgagtgtatcaggtcctggtccccaaggtccgagcggtcccgggccccaagggccatacgggccgggcccacaggggccaggcccacgtggccctggtccccaagggccaggaccgcaaggtccaggcccacagggaccgagcggcccgggcccgcaggggccgtacggaccaggcggagtcagcgttgttagcgccagcgtgtccgggccagggccacaagggccgtctggtcccatgcctcaatctccttacggacctggaccacaaggtcctggaccacagggaccgggtccccaaggaccgtctggacctggtcctcagcgccctcaaggtcctggaccacaaggaccttacggtccaggtggagtttcagtggtttctgctaccgtctcgggccctggacctcaaggtcctagtggacctggaccacaaggaccatacggtcctggaccacagggcccgggtccccaaggacctggtcctcagctgcctggtcctcaaggtccttctggtccaggtcctcagggtccttacggacctggtccacagggacctggaccacagggccctggtcctcaaggaccttctggacctggtccacaacaacctcaaggacctggaccacagagaccttacggtcccggcggtgtgagcgtcgtgtccacaactgtttcgggcccaggaccacagggacctttgggcccaggagctcaagtgccatatggtcctggaccacaagttccaggtcctcaaggacctggtcctcaaggccctagtggacctggacctcaaagacctcagggtcctggtccacagggtccttacggacccggaggcgtctcggttgtcagtcagacagtgtcaggaccaggtcctcagggaccttcaggtcctggacctcaaggaccatacggacctggaccacagggtcctggtcctcagggtcctgctccacagggtccttctggtcctggtccacaaagaccccagggacctggcccacaacgtccatacggtccaggtggaatctcagtggtttctaccacagtcagcggtcctggacctcagggaccttctgctcctggaccacaaggtccatacggacctggtccacaagtgcctggtccacaaggccctggtccacaaggaccttctggtcctggtcctcaaagaccacaaggtcctggcccacagggaccttacggacccggcggtgttagcgtcgtgtcccaaacagtctcgggacccggtcctcaaggtcctagtggccctggtcctcagggaccttatggtccaggccctcaaggccctggccctcagggaccaggtccacagggtccatcgggcgctggtccacagcgtcctcaaggtcctggtcctcaagggccttatggtcccggaggcgtgtcggttgtcagtgccactgtttcaggaccaggaccacaaggaccttcaggacctggccctcaaggaccctatggcccaggcccccagggccctggtcctcagagacctgttccacaaggcccatctggtcctagacctcaacagcctcagggacctggtccacaaagacctttcggaccaggtggagtctcagctgtgtctacaactgtttttggtcctggccctcagggaccctctggcccaggtcctcagggtccctatggccctggaccccaaggcccaggaccacagggtcctggacctcaaggcccatctggacaaggtcctcagagaccatctggacctcgtcctcaaggaccttacggcccaggcggtattagcgtggtttccgctacagtgtcgggtcctggtccccaaggtcctagtggtcctggaccacaaagaccttacggacctggtcctgagggtcctggtcctcaaggtgctggacctcagggaccaggtctgcaaagaccttctggtcctggcccccagggtccttacggtcctggaccaagaggccctccacaaggaccttatggtcctggacctcagggtccaggtccccagggccctggtccccaaggacctggacctcgtggtccttctggtcctggaccccaaggaccttacggacctggtggtgtgtcagtcgtgtcagcctctgtgagcggtcccggtcctcaaggcccatcaggtcctggtccgcagggtccatacggaccaggcccccagggtcctggcccccaaggtccaggccctcagggtcctggccctcagggccctggtcctagaggaccatctggtcctggccctcaggggccttacggtcccggtggagtgtccgttgcttctgcttcggtgagtggacccggtcctcaaggcccttctggccctggcccacaaggtccctatggcccaggcccgcaaggacctggaccaagaggaccaggacctcagggccctggaccccagggtcccggtcctcagggaccatctggccctggtccccagggtccttacggaccaggcggtgttagtgttgtctcagcttctgtcagcggccctggacctcaaggaccttctggtcctgggcctcagggaccttacggcccaggtcctcaaggtcctggacctcagggtcccggcccccaaggacctggtccccagggcccaggaccaagaggaccatccggtcctggtccgcaaggaccttatggccccggaggcgtgtctgtggtttccgcctcggttagtggcccaggtccccagggtccatctggtcctggaccccagggtccgtatggtcccggtccacagggccctggtccacaagttcccggacctcaaggacctggcccccagggtccaggtcctcaaggtccctctggacctggtcctcaaggaccatatggtccaggtggagtctccgtcgtgtcagcttctgtgagcggaccaggacctcagggaccttctgggcctggccctcaaggaccttacggtcccggtcctcagggtcctggacctcgtggtccaggcccccaaggccctggtccccagggccccggacctcagggtccatctggccctggtccgcaaggtccttatggtcccggcggtgttagtgttgtctccgcctcggtcagtggcccaggaccacaaggtccttctggaccagatatcg

SEQ ID NO: 16 - DNA, 2972 bp, Bombyx mori
gctcaaagcctcatcccaatttggagtcactcaagacatccttgattaaggcagctgccgatattgacatggacctcgttcgtgctgcgatagacgactggccgcgcagattgaaggcctgtattcaaaatcacggaggtcattttgaataaactttagtgtcataagaatctatgttttgttaagttcattttggtatatgaatggttacataatgaataaacttgtttcaattattttacattaaacatgtgacagaatttatgacctgactaggtaggtacaaacagcctttttgatattagaaaactaagtaaaatagcctacggtcacatctctttccgtgggtgtcgttaaagggcgacttagagaaccaccaagaacgtagcagaatcctcagagtgtcataccagcatacagccatcgctaactgctatttactggtaatagggcacattgtaatctcacttaaccatactgtcgggccaccatctagcctatttctgccacgaatcaatcgtgagtgatggacatagagaaactattagttgagaagaaaacaagagcactaaaggtttgatattgacaaaaatctacttcgccgtcactccataggtttattgtctctcattagtccagaacagcagttacagacgtaatcttttacgcacaaactacagggttgctctttattgtatcgaaaatatgggacctgaataagggcgattttgacgcgtcctgcccgcccattcccgatcctacggacagaatggcaagcagtcgacgtcgccccaaacacgtcatttcggctcctcacgatccactaacggtgctttaggtacctcaagcaccggtcatcgttctcgtcggacccgtcgcttgcgacgaagggctcgacgagcaaattaaccctcagacacagcccactgagtttctcgccggatcttctcagcgggtcgcgtttccgatccggtggtagattctgcgaagcacggctcttgctaggattcgtgttagcaacgtcgtcaggtttgagccccgtgagctcacttactagttaaggttacgctgaaatagcctctcaaggctctcagctaggtaggaaacaaaaaaaaaagtcctgcccttaacaccgttgcgatggcttgtcttctgcaccgcggaaagatgttttgtacggaaagtttgaataagtgcttaattgcaagtaacgtaacaatgttttagggttcggcggccgcgggagaaagcatgaagtaagttctttaaatattacaaaaaaattgaacgatattataaaattctttaaaatattaaaagtaagaacaataagatcaattaaatcataattaatcacattgttcatgatcacaatttaatttacttcatacgttgtattgttatgttaaataaaaagattaatttctatgtaattgtatctgtacaatacaatgtgtagatgtttattctatcgaaagtaaatacgtcaaaactcgaaaattttcagtataaaaaggttcaactttttcaaatcagcatcagttcggttccaactctcaagatgagagtcaaaacctttgtgatcttgtgctgcgctctgcatgtgagttaattattttactattatttcagaaggtggccagacgacatcacgggccacctgataataagtggtcgccaaaacgcacagacatcgtaaattgtgccatttgatttgtcacgccctgggggctacggaataaactacatttatttatttaaaaaatgaaccttagattatgtaacttgtgatttatttgcgtcaaaagtaggcaagatgaatctatgtaaatacctgggcagacttgcaatatcctatttcaccggtaaatcagcattgcaatatgcaatgcatattcaacaatatgtaaaacaattcgtaaagcatcattagaaaatagacgaaagaaattgcataaaattataaccgcattattaatttattatgacatctattaacaattgctattgcctttttttcgcaaattataatcattttcataacctcgaggtagcattctgttacattttaatacattggtatgtgattataacacgagctgcccactgagtttctcgccagatcttctcagtgggtcgcgttaccgatcacgtgatagattctatgaagcactgctcttgttagggctagtgttagcaaattctttcaggttgagtctgagagctcacctacccatcggagcgtagctggaataggctaccagctaataggtagggaaaacaaagctcgaaacaagctcaagtaataacaacataatgtgaccataaaatctcgtggtgtatgagatacaattatgtactttcccacaaatgtttacataattagaatgttgttcaacttgcctaacgccccagctagaacattcaattattactattaccactactaaggcagtatgtcctaactcgttccagatcagcgctaacttcgattgaatgtgcgaaatttatagctcaatattttagcacttatcgtattgatttaagaaaaaattgttaacattttgtttcagtatgtcgcttatacaaatgcaaacatcaatgattttgatgaggactattttgggagtgatgtcactgtccaaagtagtaatacaacagatgaaataattagagatgcatctggggcagttatcgaagaacaaattacaactaaaaaaatgcaacggaaaaataaaaaccatggaatacttggaaaaaatgaaaaaatgatcaagacgttcgttataaccacggattccgacggtaacgagtccattgtagaggaagatgtgctcatgaagacagttatgttgctgctgatgcgggagcatattctcagagcgggccatacgtatcaaacagtggatacagcactcatcaaggatatacgagcgatttcagcactagtgctggcatcatctagacccggg

SEQ ID NO: 17 - DNA, 2990 bp, Bombyx mori
gctcaaagcctcatcccaatttggagtcactcaagacatccttgattaaggcagctgccgatattgacatggacctcgttcgtgctgcgatagacgactggccgcgcagattgaaggcctgtattcaaaatcacggaggtcattttgaataaactttagtgtcataagaatctatgttttgttaagttcattttggtatatgaatggttacataatgaataaacttgtttcaattattttacattaaacatgtgacagaatttatgacctgactaggtaggtacaaacagcctttttgatattagaaaactaagtaaaatagcctacggtcacatctctttccgtgggtgtcgttaaagggcgacttagagaaccaccaagaacgtagcagaatcctcagagtgtcataccagcatacagccatcgctaactgctatttactggtaatagggcacattgtaatctcacttaaccatactgtcgggccaccatctagcctatttctgccacgaatcaatcgtgagtgatggacatagagaaactattagttgagaagaaaacaagagcactaaaggtttgatattgacaaaaatctacttcgccgtcactccataggtttattgtctctcattagtccagaacagcagttacagacgtaatcttttacgcacaaactacagggttgctctttattgtatcgaaaatatgggacctgaataagggcgattttgacgcgtcctgcccgcccattcccgatcctacggacagaatggcaagcagtcgacgtcgccccaaacacgtcatttcggctcctcacgatccactaacggtgctttaggtacctcaagcaccggtcatcgttctcgtcggacccgtcgcttgcgacgaagggctcgacgagcaaattaaccctcagacacagcccactgagtttctcgccggatcttctcagcgggtcgcgtttccgatccggtggtagattctgcgaagcacggctcttgctaggattcgtgttagcaacgtcgtcaggtttgagccccgtgagctcacttactagttaaggttacgctgaaatagcctctcaaggctctcagctaggtaggaaacaaaaaaaaaagtcctgcccttaacaccgttgcgatggcttgtcttctgcaccgcggaaagatgttttgtacggaaagtttgaataagtgcttaattgcaagtaacgtaacaatgttttagggttcggcggccgcgggagaaagcatgaagtaagttctttaaatattacaaaaaaattgaacgatattataaaattctttaaaatattaaaagtaagaacaataagatcaattaaatcataattaatcacattgttcatgatcacaatttaatttacttcatacgttgtattgttatgttaaataaaaagattaatttctatgtaattgtatctgtacaatacaatgtgtagatgtttattctatcgaaagtaaatacgtcaaaactcgaaaattttcagtataaaaaggttcaactttttcaaatcagcatcagttcggttccaactctcaagatgagagtcaaaacctttgtgatcttgtgctgcgctctgcatgtgagttaattattttactattatttcagaaggtggccagacgacatcacgggccacctgataataagtggtcgccaaaacgcacagacatcgtaaattgtgccatttgatttgtcacgccctgggggctacggaataaactacatttatttatttaaaaaatgaaccttagattatgtaacttgtgatttatttgcgtcaaaagtaggcaagatgaatctatgtaaatacctgggcagacttgcaatatcctatttcaccggtaaatcagcattgcaatatgcaatgcatattcaacaatatgtaaaacaattcgtaaagcatcattagaaaatagacgaaagaaattgcataaaattataaccgcattattaatttattatgacatctattaacaattgctattgcctttttttcgcaaattataatcattttcataacctcgaggtagcattctgttacattttaatacattggtatgtgattataacacgagctgcccactgagtttctcgccagatcttctcagtgggtcgcgttaccgatcacgtgatagattctatgaagcactgctcttgttagggctagtgttagcaaattctttcaggttgagtctgagagctcacctacccatcggagcgtagctggaataggctaccagctaataggtagggaaaacaaagctcgaaacaagctcaagtaataacaacataatgtgaccataaaatctcgtggtgtatgagatacaattatgtactttcccacaaatgtttacataattagaatgttgttcaacttgcctaacgccccagctagaacattcaattattactattaccactactaaggcagtatgtcctaactcgttccagatcagcgctaacttcgattgaatgtgcgaaatttatagctcaatattttagcacttatcgtattgatttaagaaaaaattgttaacattttgtttcagtatgtcgcttatacaaatgcaaacatcaatgattttgatgaggactattttgggagtgatgtcactgtccaaagtagtaatacaacagatgaaataattagagatgcatctggggcagttatcgaagaacaaattacaactaaaaaaatgcaacggaaaaataaaaaccatggaatacttggaaaaaatgaaaaaatgatcaagacgttcgttataaccacggattccgacggtaacgagtccattgtagaggaagatgtgctcatgaagacactttccgatggtactgttgctcaaagttatgttgctgctgatgcgggagcatattctcagagcgggccatacgtatcaaacagtggatacagcactcatcaaggatatacgagcgatttcagcactagtgctgtagtccccggg

SEQ ID NO: 18 - DNA, 537 bp, Bombyx mori
gtccgggaagcgtcagttacggagctggcaggggatacggacaaggtgcaggaagtgcagcttcctctgtgtcatctgcttcatctcgcagttacgactattctcgtcgtaacgtccgcaaaaactgtggatttcctagaagacaactagttgttaaattcagagcactgccttgtgtgaattgctaatttttaatataaaataacccttgtatgttttttttttcgttaataaatgagagcatttaagttattgtttttaattacttttttttagaaaacagatttcggattttttgtatgcattttatttgaatgtactaatataatcaattaatcaatgatttcatttatttaagggataacaataatccatgatttcacatgcacatttaaaacaaaactaaattacaataggttcatataaaaacaacaagtatgccttctcaactaagaatactatagggatccacaaaactaaattacaataggttcatataaaaacaacaagtatgccttctcaactaagaatactata

SEQ ID NO: 19 - DNA, 1016 bp, Bombyx mori
ttcaaaacaattcgtaaagcatcattagaaaatagacgaaagaaattgcataaaattataaccgcattattaatttattatgatttgtattaacaattgctattgcctttttttcgcaaattataatcattttcataacctcgaggtagcattctgttacattttaatacattggtatgtgattataacacgagctgcccactgagtttctcgccagatcttctcagtgggtcgcgttaccgatcacgtgatagattctatgaagcactgctcttgttagggctagtgttagcaaattctttcaggttgagtctgagagctcacctacccatcggagcgtagctggaataggctaccagctaataggtagggaaaacaaagctcgaaacaagctcaagtaataacaacataatgtgaccataaaatctcgtggtgtatgagatacaattatgtactttcccacaaatgtttacataattagaatgttgttcaacttgcctaacgccccagctagaacattcaattattactattaccactactaaggcagtatgtcctaactcgttccagatcagcgctaacttcgattgaatgtgcgaaatttatagctcaatattttagcacttatcgtattgatttaagaaaaaattgttaacattttgtttcagtatgtcgcttatacaaatgcaaacatcaatgattttgatgaggactattttgggagtgatgtcactgtccaaagtagtaatacaacagatgaaataattagagatgcatctggggcagttatcgaagaacaaattacaactaaaaaaatgcaacggaaaaataaaaaccatggaatacttggaaaaaatgaaaaaatgatcaagacgttcgttataaccacggattccgacggtaacgagtccattgtagaggaagatgtgctcatgaagacactttccgatggtactgttgctcaaagttatgttgctgctgatgcgggagcatattctcagagccccgggtctaga

SEQ ID NO: 20 - DNA, 504 bp, Bombyx mori
ttgtttaaaccgtaaaaaaagtcatatttctgtatatcaaaacacatctaatattaaaaaaacagtcagcaagcacttacaagtgtgggctcggacagcaattacctggtctcaggagacacttgaagaacgagaagcacgtctctctgtcgattgcgaggctcatgcactatcgcttgagtctgagacctttactgatagggaaatccgtttgagctctcagagggttcggacagcaaaggcttgcccggtctcaggagacattagaagaacgggaagcataactcaataccgatcgcgtttccattgagcctatgcttcgtgataataataaataaagcccaaggtcagacgctaaaagttgtgggtgtagatataagtgttaactgcttttcacacagtcaactcggtcacgcggttatgtagctctgtcacatgctagcaatcctagcaacctgtatgctttgatccccgatggctatacgtctacgtcgtgtatagaga

SEQ ID NO: 21 - DNA, 937 bp, Bombyx mori
gcaggagctggtgcaggtgctgccgctggttctggtgcgggtgccggagctggttatggagctgcttctggtgctggtgccggtgctggggctggtgccggagctggttatggaactggtgcaggtgcaggtgccggagctggttatggagctggtgcaggtgcaggtgccggagctggttatggggctggtgcaggtgcaggtgccggagctggttatggagctggtgcaggtgcaggtgccggagctggttatggggctggtgcaggtgcaggtgccggagctggttatggagctggtgcgggtgccggtgccggggctggttatggagctgcctctggtgctggtgctggcgctgggtacggacaaggagtaggaagcggagctgcttctggagctggtgcaggtgcaggagcaggttctgccgctggttctggggcaggtgccggtgctggtaccggtgctggtgcaggttacggagctggtgcaggtgccggtgccggagctggttatggagctgcctctggtactggagcaggttatggagctggtgccggagctggttacggaggtgcctctggtgctggtgctggtgccggtgctggggctggagccggtgctggtgcaggttatggaactggcgctggatacggagcaggagccggagcaggagccggagcaggagctggtgctggatacggagcaggagctggtgctggatacggagcaggatatggagtaggagctggtgctggatacggagcaggatacggagcaggagctggaagcggagctgcctctggtgctggttcaggtgccggtgctggttcaggtgccggtgctggttcaggtgccggtgctggttcaggtgccggtgccggttcaggtgctggtgctggttcaggtgctggtgctggttcaggtgctggtgcaggtt

(19) United States
(12) Patent Application Publication
(10) Pub. No.: US 2021/0246471 A1
(43) Pub. Date: Aug. 12, 2021

(54) MODIFICATION OF HEAVY CHAIN FIBROIN IN BOMBYX MORI

(71) Applicant: Kraig Biocraft Laboratories, Inc., Ann Arbor, MI (US)

(72) Inventors: Trevor Logan Kane, Dewitt, MI (US); Ana Laura Licon, Brighton, MI (US); Kimberly Kraig Thompson, East Lansing, MI (US)

(21) Appl. No.: 17/172,818

(22) Filed: Feb. 10, 2021

Related U.S. Application Data
(60) Provisional application No. 62/995,717, filed on Feb. 11, 2020.

(51) Int. Cl. C12N 15/90; A01K 67/04; C12N 15/11; C12N 9/22; C07K 14/435

(52) U.S. Cl. CPC C12N 15/902; A01K 67/04; C12N 15/11; C12N 9/22; C07K 14/43518; A01K 2217/075; C12N 2800/80; A01K 2267/01; A01K 2227/706; A01K 2217/054; C12N 2310/20

(57) ABSTRACT

Described herein are methods of producing transgenic Bombyx mori by targeting and modifying genomic regions associated with the heavy chain fibroin protein. Embodiments include insertion and truncation vectors utilized for modifying the FibH gene. Embodiments include plasmid constructs utilized for molecular cloning of donor sequences configured for replacement of or insertion into the FibH gene and utilized for transfection of Bombyx mori with the donor sequences. Embodiments include transgenic Bombyx mori that have been transfected with the donor sequences and are capable of producing an enhanced silk product with a high percentage of spider silk proteins. Embodiments include a silk product produced by such transgenic Bombyx mori.

### CROSS-REFERENCE TO RELATED APPLICATIONS

This application claims priority to and the benefit of U.S. Provisional Patent Application Ser. No. 62/995,717, filed Feb. 11, 2020 and titled “Method for the Genetic Removal and Replacement of Heavy Chain Fibroin of Bombyx Mori”, the entirety of which is incorporated herein by this reference.

### INCORPORATION BY REFERENCE OF SEQUENCE LISTING

The file filed in conjunction with this application includes a numerical listing of sequences corresponding to the sequences (each identified by a unique SEQ ID NO) described herein. The SeqList.txt file was created on Feb. 10, 2021 and has a size of 73,389 bytes. The SeqList.txt is expressly incorporated herein by this reference.

### BACKGROUND

### Technical Field

This disclosure generally relates to methods of producing transgenic Bombyx mori (i.e., domestic silkworm) by targeting and modifying genomic regions associated with the heavy chain fibroin protein.

Related Technology

Bombyx mori is an insect from the Bombycidae moth family, most commonly referred to simply as the silkworm (in the larvae stage) or silk moth (in the adult stage). Bombyx mori were domesticated thousands of years ago in China for their ability to produce relatively large quantities of silk. Selective breeding has, over time, enabled domestic Bombyx mori to produce almost 10 times as much silk as their wild counterparts.

After a silkworm has molted four times, it will enter the pupal stage by forming a cocoon made of raw silk. The cocoon is typically formed from a single filament that can average more than 900 meters in length. The silk is harvested by steaming or boiling the cocoon before the adult moth can form and release protease enzymes, which would damage the silk of the cocoon.

Although Bombyx mori has been the most utilized silk producing organism for thousands of years, the silk produced by Bombyx mori has inferior mechanical properties when compared to silk produced by other organisms, in particular as compared to various forms of spider silk. Spider silk is believed to rank among the best of the known natural fibers due to its superior mechanical properties. However, large scale harvesting of spider silk through the raising of spiders has never been feasible due to the territorial and cannibalistic nature of spiders.

Although silk has traditionally been utilized primarily as a fabric, there are many other potential uses of the product, especially if the enhanced mechanical properties of spider silk can be realized. Examples include use as a tissue engineering scaffolding, medical implant products, ballistic-resistant material, structural material, for wound healing, and for other scenarios where the benefits of high toughness and strength to weight ratios are applicable.

There have been attempts to produce transgenic silkworms capable of expressing spider silk proteins. However, it has been challenging to transgenically produce spider silk with the desired mechanical characteristics, at appropriate scale, and in a cost-effective manner. Accordingly, there are a number of disadvantages with the conventional technology.

### SUMMARY

As discussed above, it has been challenging to produce spider silk at large scale and in a cost-effective manner in part due to the inability to culture spiders en masse for this purpose. Moreover, although there have been attempts to utilize other organisms to produce spider silk, these efforts have also met significant challenges such as the need to purify the silk and difficulty in achieving an end product with the desired mechanical properties.

Silkworms transfected with spider silk DNA is one promising approach to achieving effective and economical production of enhanced silk products. Silkworms have the inherent ability to spin silk fibers at relatively high purity levels, reducing the need for complicated downstream processing of the product. Silkworms have also been cultured for thousands of years, and a mature sericulture industry is already in place.

However, the potential of transgenic silk worms for producing enhanced silk products has yet to be realized. A major problem has been the inability to incorporate sufficient genetic changes into the silkworms to lead to the desired results. Silk proteins derive their strength from their relatively large size and their inclusion of several repeating motifs. For this same reason, however, the nucleotide sequences encoding silk proteins are large, filled with many repeats, and difficult to appropriately incorporate into the target silkworms.

As such, past approaches have been limited to relatively small insertions with limited effect on the resulting silk product. For example, the resulting silk product will typically maintain a large proportion (e.g., more than 50-60%) of the native Bombyx mori silk protein. Other significant limitations include the fact that the resulting silk product often fails to provide superior mechanical properties when compared to native silkworm silk, and the fact that even when silkworms successfully produce spider silk, the overall silk production is often significantly reduced.

Described herein are methods of producing transgenic Bombyx mori by targeting and modifying genomic regions associated with the heavy chain fibroin protein (i.e., methods for modifying the FibH gene). Embodiments include insertion and truncation vectors utilized for modifying the FibH gene. Embodiments include plasmid constructs utilized for molecular cloning of donor sequences configured for replacement of or insertion into the FibH gene and utilized for transfection of Bombyx mori with the donor sequences. Embodiments include transgenic Bombyx mori that have been transfected with the donor sequences and are capable of producing an enhanced silk product with a high percentage of spider silk proteins. Embodiments include a silk product produced by such transgenic Bombyx mori.

The donor sequences and associated vectors described herein are larger and more complex than those utilized in the prior art. Their successful use has beneficially led to transgenic Bombyx mori capable of producing a silk product with a high proportion of spider silk, with little to no negative effects on the overall production of silk from the silkworms. The resulting silk product has also demonstrated enhanced mechanical properties, particularly in regards to strength and elasticity.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIGS. 1A-1F illustrate exemplary plasmid maps including donor sequences designed for introduction into the Bombyx mori genome, the vector portion of the plasmid including homologous arms designed to enable knock-in of the donor sequence following knockout of all or a portion of the FibH gene;

FIGS. 2A and 2B illustrate exemplary plasmid maps including donor sequences designed for introduction into the Bombyx mori genome in order to modify or replace the FibH gene, the vector portion of the plasmids including homologous arms designed to enable insertion of the donor sequence into the native FibH gene;

and

FIG. 3 is a Western blot gel showing that proteins derived from transgenic Bombyx mori include the A2S8 protein, indicating successful incorporation of the spider silk protein into the Bombyx mori genome at the targeted FibH gene and successful production of an enhanced silk product therefrom.

### DETAILED DESCRIPTION

### Introduction

With respect to various terms of art and molecular biology details disclosed herein, reference is made to Sambrook, Fritsch, Maniatis, Molecular Cloning, A LABORATORY MANUAL (2d Edition, Cold Spring Harbor Laboratory Press, 1989) (especially Volume 3), and Kendrew, THE ENCYCLOPEDIA OF MOLECULAR BIOLOGY (Blackwell Science Ltd 1995. When combined with the teachings of this disclosure, the teachings of these references can be suitably modified, without undue experimentation, to enable the skilled artisan to utilize molecular biology techniques to construct the various vectors disclosed herein, to clone vectors into suitable plasmids, and to transfect and form recombinant Bombyx mori.

Heavy Fibroin Target

Bombyx mori silk is made up of two major components: fibroin and sericin. Fibroin is produced in heavy chain, light chain, and glycoprotein P25 form. When the silkworm produces silk, the heavy and light chains are linked by disulphide bonding, and the P25 integrates via non-covalent interactions. The sericin proteins are hydro-soluble and function to coat and adhere separate fibroin filaments as the silkworm generates the silk. In commercial silk production, the sericin is typically removed as an unimportant side product.

Although the fibroin heavy chain and the fibroin light chain are included in the silk in approximately the same molar ratio, the fibroin heavy chain has a much higher molecular weight than the light chain (about 350 kDa compared to about 26 kDa). The fibroin heavy chain thus makes up the majority of Bombyx mori silk. Targeting the fibroin heavy chain gene (FibH) for modification therefore allows significant changes to the mechanical properties of the silk generated by the resulting transgenic silkworm.

As used herein, “modification” of the fibroin heavy chain gene includes embodiments where the entire FibH gene, or one or more portions thereof, are knocked out of the silkworm genome and replaced with a knock-in insert (e.g., using a truncation vector). The term also includes embodiments where one or more inserts are inserted at a position within the FibH gene and/or at a position functionally adjacent to the gene (e.g., using an insertion vector). Some knockout embodiments are configured to knockout exon2 (about 16 kbp) of the FibH gene. In some knockout embodiments, at least about 50% of the FibH gene is knocked out, or at least about 60%, or at least about 70%, or at least about 80% of the FibH gene is knocked out.

In some embodiments, the FibH gene is targeted in a manner that retains the native FibH promoter within the genome. As a result, the resulting transgenic silkworm is able to utilize the native promoter for expression of the knocked-in insert. Alternative embodiments target the FibH promoter for inclusion in the knockout portion, and include one or more separate promoter sequences as part of the knock-in insert.

A relevant section of the FibH gene is provided as SEQ ID NO:1. Minor variations of the gene can occur due to differences in particular silkworm varieties, and the disclosed sequence is exemplary only. The skilled person will understand that the principles and components described herein can be utilized with other variations of the FibH gene with only minor or no modification required.

Exemplary DNA targets associated with specific locations of the FibH gene for targeting by guide RNAs (gRNAs) are provided as SEQ ID NO:2 to SEQ ID NO:8. In particular, SEQ ID NO:2 and/or SEQ ID NO:3 may be utilized as targets for upstream gRNAs, and SEQ ID NO:4 and/or SEQ ID NO:5 may be utilized as targets for downstream gRNAs, in a Mad7 system in which the PAM sequence is YTTN. In another embodiment, SEQ ID NO:6 and/or SEQ ID NO:7 may be utilized as targets for upstream gRNAs, and SEQ ID NO:8 may be utilized as a target for a downstream gRNA, in a CRISPR/Cas9 system in which the PAM sequence is NGG. The locations of the FibH gene in which these exemplary gRNAs targets can be found by matching the gRNA target locations (or in most instances, their reverse complements) to the corresponding location in the FibH gene (e.g., as provided by SEQ ID NO:1). In some embodiments, reverse complements of one or more of the foregoing may additionally or alternatively be utilized as suitable gRNA targets.

The particular portions of the FibH gene targeted for knockout and/or as an insertion site will vary somewhat depending on the particular gene editing process utilized. This is a result of inherent differences in gene editing techniques. For example, the standard Cas9 nuclease requires a protospacer adjacent motif (PAM) with sequence NGG, whereas the standard Mad7 endonuclease requires a PAM with sequence YTTN. Other Cas9, Mad7, or other gene editing nucleases may have other associated PAM sequences, and thus the corresponding gRNAs may be varied accordingly. Other gene editing techniques such as those using transcription activator-like effector nucleases (TALENs) or zinc finger nucleases (ZFNs) have other inherent characteristics that must be accounted for when selecting a particular target site within the FibH gene. The skilled person, in light of the teachings of this disclosure, is able to determine appropriate FibH targets for these other gene editing processes.

Spider Silk Insert

As mentioned above, many spider silks provide superior mechanical properties and would be beneficial for a variety of applications. However, cost-effective and appropriately scaled production of such silks has been elusive due to the technical challenges involved with producing the silk. The vectors and related methods described herein include spider silk protein sequences that enable the resulting transgenic silkworms to produce an enhanced silk product with beneficially enhanced mechanical properties.

Examples of sequences encoding spider silk proteins that can be included in the donor insert include those related to the proteins MaSp2, flagelliform, A2S8 (which includes alternating repeating motifs of MaSp2 and flagelliform), MaSp1, MaSp4, and MiSp. Although these proteins are presently preferred, other spider protein sequences may additionally or alternatively be included. Particularly preferred sequences are those associated with tangle-web weaver spiders (e.g., Latrodectus hesperus) and orb-weaver spiders such as golden orb-weaver spiders (Nephila) and the Darwin's bark spider (Caerostris darwini). These types of spiders in general, and the Darwin's bark spider in particular, can produce silk with extremely beneficial mechanical properties.

In some embodiments, a single spider silk protein-encoding sequence is included in the donor insert in order to provide a single spider silk protein. In other embodiments, the donor insert includes sequences that encode two or more spider silk proteins. For example, a donor insert may include a set of sequences that encodes two or more of A2S8, MaSp1, and MaSp4. Donor inserts may additionally or alternatively include repeated sequences that would each separately encode the same protein. For example, a sequence that encodes for a particular spider silk protein may be repeated multiple times within the insert in order to provide a translated protein that is multiple times longer than the native protein, thereby providing differences in mechanical properties.

Examples of effective sequences that encode for A2S8 are provided by SEQ ID NO:9 and SEQ ID NO:10. The A2S8 protein is a combination of alternating repeating motifs of MaSp2 and flagelliform that beneficially provides effective strength and elasticity.

Examples of effective sequences that encode for MaSp1 (Caerostris darwini) are provided by SEQ ID NO:11 and SEQ ID NO:12, with another exemplary form of MaSp1 (Nephila clavipes) provided by SEQ ID NO:13. Examples of effective sequences that encode for MaSp4 (Caerostris darwini) are provided by SEQ ID NO:14 and SEQ ID NO:15.

In some embodiments, the donor insert can further include an N-terminal domain (NTD) and/or a C-terminal domain (CTD) sequence. The NTD and CTD are native sequences of the silkworm FibH gene. The inclusion of the NTD and/or CTD promotes better association of the translated protein with other Bombyx mori proteins. In particular, the NTD and/or CTD sequences enable the translated protein to better integrate with the light chain fibroin and the P25 proteins, which is beneficial in certain applications where such integration is desired. In some embodiments, the target for nuclease activity is located within or downstream of the native NTD. In such embodiments, an NTD included as part of the donor sequence can be utilized to further guide the donor sequence and ensure that it the remaining native portions of the FibH gene stay in frame with the inserted donor sequence.

In other embodiments, the NTD and/or CTD sequence is/are intentionally omitted from the insert. Although there are certain benefits to their inclusion, they are part of the native silkworm FibH gene, and omitting them allows for the generation of a silk product with a higher proportion of spider silk. Thus, in some applications where higher purity and higher proportion of spider silk proteins are desired, omitting one or both of the NTD and CTD sequences is beneficial. Where the NTD and/or CTD are omitted, the target sites for knockout of the FibH gene can be tailored such that only the minimal required portions of the native NTD and CTD remain in the genome.

Exemplary NTD sequences are provided by SEQ ID NO:16 and SEQ ID NO:17, and an exemplary CTD sequence is provided by SEQ ID NO:18. These sequences may be varied to some extent based on the particular Bombyx mori variants utilized, through modification of end portions that transition between the terminal domain sequences and the spider silk sequence(s), and/or through modification of end portions that transition between the terminal domain sequences and homologous arm sequences, for example.

In some embodiments, the donor DNA insert may additionally include protein-encoding sequences that enable translation of fusion proteins. The spider silk may, for example, be fused with a reporter, such as luciferase, or with an N- or C-terminal epitope tag, such as FLAG, 6X-His, or other epitope tag known to those having skill in the art.

Gene Editing Methods

Various gene editing methods may be utilized to target and modify the FibH gene of Bombyx mori. Most gene editing methods rely on targeted endonuclease activity, and vary based on the particular endonuclease utilized and the corresponding targeting technique inherent to the endonuclease used. ZFNs or TALENs may be utilized, but are typically less preferred due to the necessity of designing and constructing new for each target. Presently, more preferred methods include those that utilize clustered regularly interspaced short palindromic repeats (CRISPR) methods, including those that make use of the Mad7 nuclease or the Cas9 nuclease, for example.

The choice of gene editing process utilized to form the transgenic Bombyx mori affects the design of gRNAs and/or the particular portion of the FibH gene targeted for nuclease activity. That is, particular portions of the FibH gene targeted for knockout and/or as an insertion site will vary somewhat depending on the particular gene editing process utilized due to inherent differences in the target requirements and activity of the different nucleases (e.g., different PAM sequence requirements).

Although the particular examples of gRNAs and vectors described herein are designed for Cas9 and Mad7 systems, other gRNAs and/or target sites may be utilized where other gene editing systems are used. Although the particular target site of the FibH gene may vary with different systems and/or with different gRNAs in order to accommodate different nuclease functionality, the target should preferably still be within about 200 base pairs, more preferably about 100 base pairs, even more preferably within about 50 base pairs, of the target site when the disclosed gRNAs and their corresponding nuclease systems are utilized.

Vector Construction

Vectors utilized to modify the FibH gene include one or more spider silk sequences, such as one or more of the sequences provided by SEQ ID NO:9 to SEQ ID NO:15. The vectors may also include an NTD and/or CTD adjacent the spider silk sequences, such as the NTD and CTD sequences provided by SEQ ID NO:16 through SEQ ID NO:18.

The vectors also include homologous arms designed to guide insertion of the donor sequence(s) into the targeted portion of the FibH gene. The form of the homologous arms will vary depending on the particular site of the FibH gene targeted for nuclease activity. The homologous arms are designed to have sufficient homology to the remaining upstream and downstream portions of the Bombyx mori genome following nuclease activity in order to guide appropriate insertion via homology directed repair.

An exemplary upstream homologous arm for a truncation vector as disclosed herein is provided by SEQ ID NO:19. An exemplary downstream homologous arm for a truncation vector as disclosed herein is provided by SEQ ID NO:20. These and similar homologous arms are suitable for introducing the donor sequence into the genome following knockout of the portions of the FibH gene natively residing between the homologous arms.

For an insertion vector, an exemplary upstream homologous arm can be the same as utilized in a truncation vector, such as SEQ ID NO:19. An exemplary downstream homologous arm for a truncation vector as disclosed herein is provided by SEQ ID NO:21. The downstream homologous arm for an insertion vector corresponds to a sequence located close to the upstream homologous arm in the native sequence. This minimizes knockout and instead promotes insertion of the donor sequence into the FibH gene in addition to the native protein encoding sequences.

These exemplary homologous arm sequences may be varied somewhat, the downstream portion of the upstream homologous arm and the upstream portion of the downstream homologous arm in particular, to account for differences in FibH gene variants and/or different nuclease target sites as appropriate.

The use of a truncation vector versus an insertion vector involves different tradeoffs, and one may be preferred over another depending on particular application needs. For example, a truncation vector provides a resulting silk product with a higher proportion of spider silk due to the removal of much of the native silk encoding sequences. On the other hand, an insertion vector adds to the overall size of the resulting silk proteins, which can beneficially affect mechanical properties of the silk.

The donor sequences described herein are much larger than donor sequences utilized in prior Bombyx mori vectors. For example, a vector may include a donor sequence portion (i.e., the portion not including homologous arms) of greater than about 2 kbp, or greater than about 4 kbp, or greater than about 6 kbp, or greater than about 8 kbp, or greater than about 10 kbp, or greater than about 12 kbp, or greater than about 14 kbp, greater than about 16 kbp, or greater than about 18 kbp. A donor sequence may therefore range in size from about 2 kbp to about 20 kbp, though other ranges utilizing any two of the foregoing values as endpoints may also be utilized.

The large relative size of the donor sequence portion allows for a large resulting silk protein and the concomitant benefits to mechanical properties associated therewith. The size of the donor sequence portion is also large relative to the homologous arms used to guide insertion, and yet, surprisingly, the exemplified donor sequences were able to be successfully introduced into the FibH gene. The homology arms are typically about 500 bp to 1 kbp, for example. Typically, the donor sequence insert is approximately the same size as the homology arms. Here, however, the disclosed vectors proved effective even though the donor sequence portion can be more than 2 times the size of the average size of the homology arms. More typically, the donor sequence is more than 5 times, more than 8 times, more than 12 times, more than 16 times, more than 20 times, more than 24 times, more than 28 times, more than 32 times, more than 36 times, or more than 40 times the average size of the homology arms. A donor sequence may therefore be about 2 to about 40 times the average size of the corresponding homology arms. For example, an insert of about 20 kbp may be paired with homology arms of about 500 bp. Other ranges utilizing any two of the foregoing values as endpoints may also be utilized.

The following non-exhaustive list of examples of vectors can utilize the sequences identified and disclosed herein, including those of SEQ ID NO:9 to SEQ ID NO:21, for one or more of their listed components, including the sequences for homologous arms, spider silk sequences, and NTD and CTD sequences.

An exemplary vector based on the Mad7 system and associated FibH target sites includes an upstream homologous arm, an NTD, an A2S8 spider silk sequence, a CTD, and a downstream homologous arm suitable for truncation of the FibH gene, or alternatively a downstream homologous arm suitable for insertion of the donor insert into the FibH gene.

An exemplary vector based on the Mad7 system and associated FibH target sites includes an upstream homologous arm, an NTD, a MaSp1 (Caerostris darwini) spider silk sequence, a CTD, and a downstream homologous arm suitable for truncation of the FibH gene, or alternatively a downstream homologous arm suitable for insertion of the donor insert into the FibH gene.

An exemplary vector based on the Mad7 system and associated FibH target sites includes an upstream homologous arm, an NTD, a MaSp4 (Caerostris darwini) spider silk sequence, a CTD, and a downstream homologous arm suitable for truncation of the FibH gene, or alternatively a downstream homologous arm suitable for insertion of the donor insert into the FibH gene.

An exemplary vector based on the Cas9 system and associated FibH target sites includes an upstream homologous arm, an NTD, a MaSp1 (Caerostris darwini) spider silk sequence, a CTD, and a downstream homologous arm suitable for truncation of the FibH gene, or alternatively a downstream homologous arm suitable for insertion of the donor insert into the FibH gene.

An exemplary vector based on the Mad7 system and associated FibH target sites includes an upstream homologous arm, a MaSp1 (Caerostris darwini) spider silk sequence provided in two or more sequential sets, a MaSp4 (Caerostris darwini) spider silk sequence provided in two or more sequential sets, and a downstream homologous arm suitable for truncation of the FibH gene, or alternatively a downstream homologous arm suitable for insertion of the donor insert into the FibH gene.

An exemplary vector based on the Mad7 system and associated FibH target sites includes an upstream homologous arm, a MaSp1 (Caerostris darwini) spider silk sequence provided in two or more sequential sets, a MaSp4 (Caerostris darwini) spider silk sequence provided in two or more sequential sets, an A2S8 spider silk sequence, a MaSp1 (Nephila clavipes) spider silk sequence, and a downstream homologous arm suitable for truncation of the FibH gene, or alternatively a downstream homologous arm suitable for insertion of the donor insert into the FibH gene.

An exemplary vector based on the Mad7 system and associated FibH target sites includes an upstream homologous arm, an A2S8 spider silk sequence, and a downstream homologous arm suitable for truncation of the FibH gene, or alternatively a downstream homologous arm suitable for insertion of the donor insert into the FibH gene.

An exemplary vector based on the Cas9 system and associated FibH target sites includes an upstream homologous arm, an A2S8 spider silk sequence, and a downstream homologous arm suitable for truncation of the FibH gene, or alternatively a downstream homologous arm suitable for insertion of the donor insert into the FibH gene.

Plasmid Construction & Cell Transfection

The vectors described herein may be inserted into plasmids. The plasmids may include features known in the art for enabling cloning and amplification. The plasmid may include an origin of replication, a suitable site for cloning (e.g., a multiple cloning site), a selection gene (e.g., ampicillin resistance), various regulatory sequences (e.g., promoters, binding sites, lac promoter and operon, etc.), and primer sites, for example. Various plasmid backbones are known in the art and are suitable for use with the vectors described herein. Examples include pUC57 and other plasmids of similar function and ability to receive vectors with the sizes disclosed herein.

In some embodiments, a plasmid can include the vector sequence as well as a sequence encoding for the nuclease (e.g., Cas9 or Mad7) of the associated gene editing process intended for incorporating the vector into the FibH gene. However, presently preferred embodiments deliver the nuclease and corresponding gRNAs separately in order to improve delivery and incorporation into the genome by preventing the plasmids from becoming too large.

Plasmids may be delivered to the target Bombyx mori cells via one or more suitable transfection methods known in the art. For example, silkworm eggs may be transfected via microinjection, electroporation, other transfection method, or combinations thereof. Following transfection, the plasmids may be linearized using targeted restriction enzymes and/or other known methods. In some embodiments, a nuclease target site associated with the nuclease of the corresponding gene editing method may be cloned into the plasmid such that the plasmid is itself targeted and linearized by the same nuclease used to target the host genome.

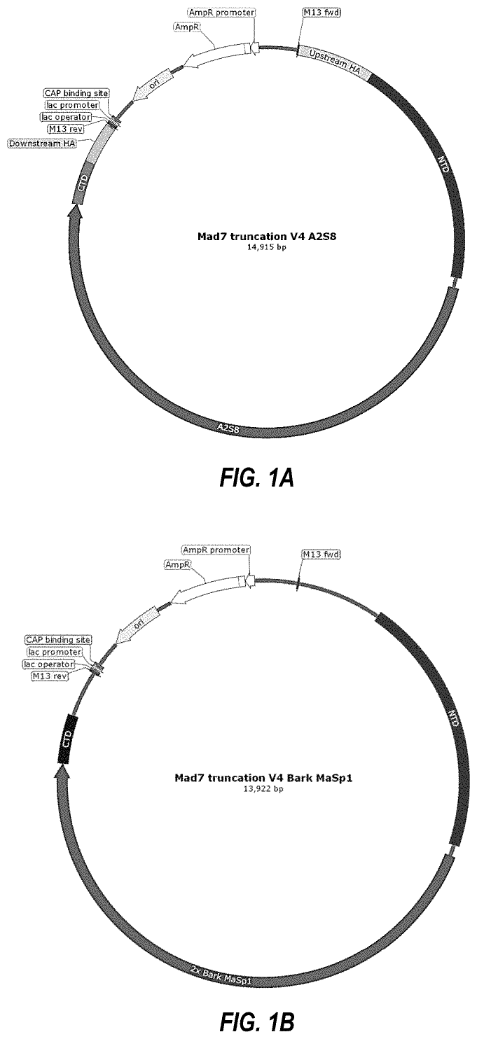

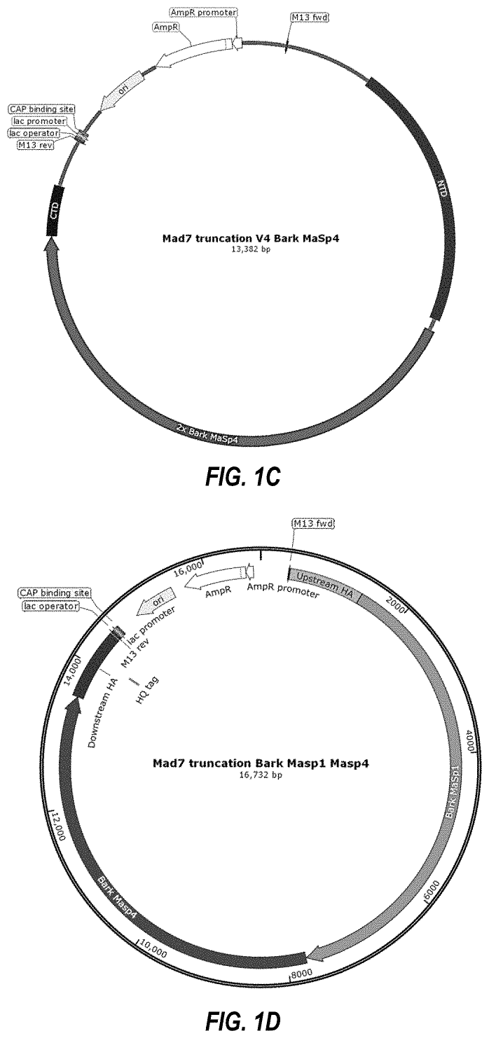

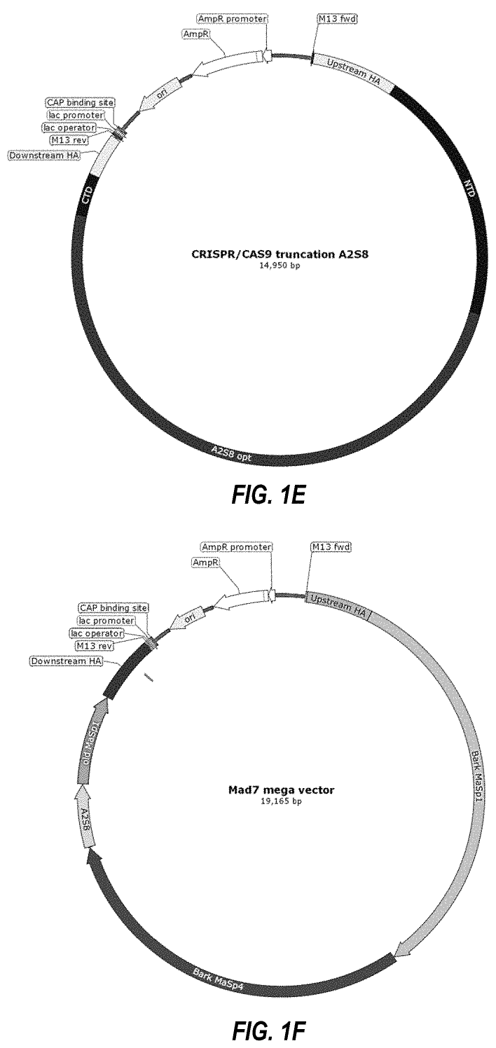

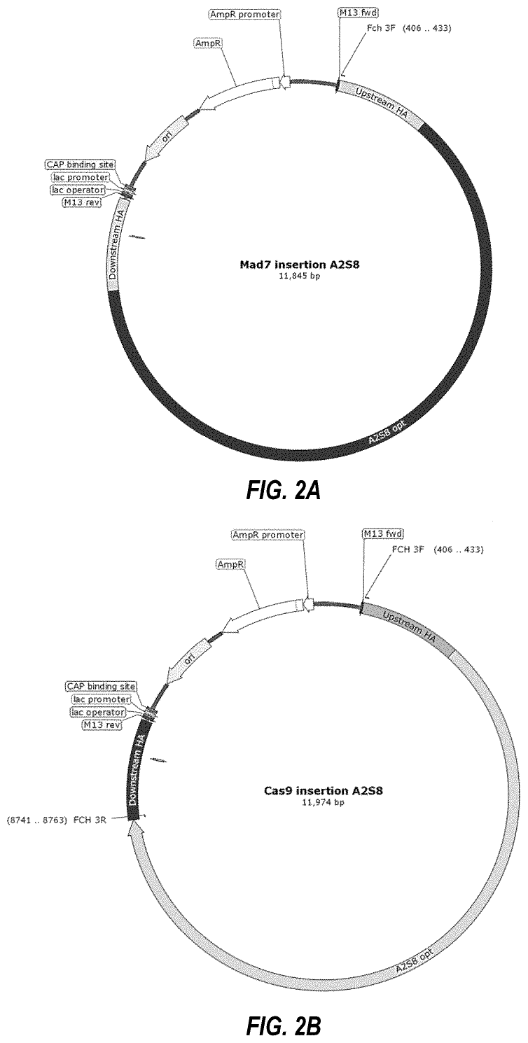

FIGS. 1A-1F illustrate exemplary plasmid maps that include donor sequences designed for introduction into the Bombyx mori genome, the vector portion of the plasmid including homologous arms designed to enable knock-in of the donor sequence following knockout of all or a portion of the FibH gene. FIGS. 2A and 2B illustrate exemplary plasmid maps that include donor sequences designed for introduction into the Bombyx mori genome in order to modify or replace the FibH gene, the vector portion of the plasmids including homologous arms designed to enable insertion of the donor sequence into the native FibH gene. Note that some of the illustrated vectors include NTD and CTD sequences, while others do not. In some of the vectors, the NTD and CTD sequences can be utilized as the homologous arms.

Silk Product Examples

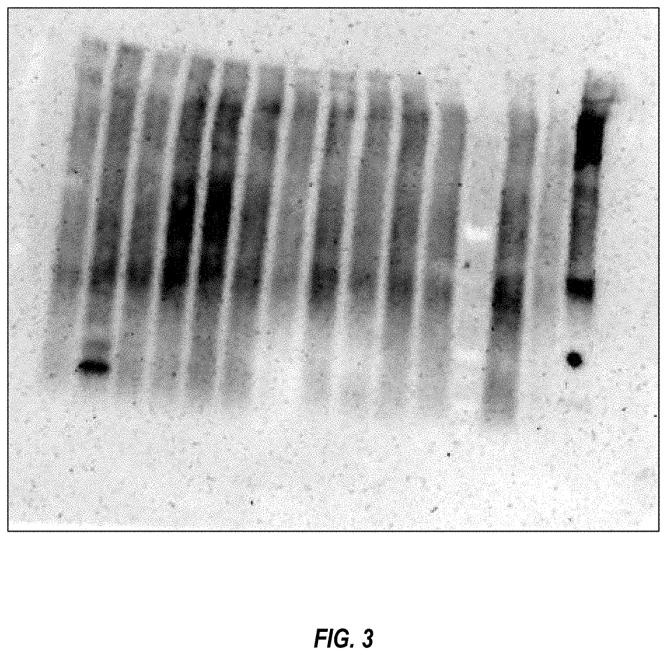

FIG. 3 is a Western blot gel showing that proteins derived from transgenic Bombyx mori include the A2S8 protein, indicating successful incorporation of the spider silk protein into the Bombyx mori genome at the targeted FibH gene and successful production of an enhanced silk product therefrom. In the Figure, the right lane is the positive control, the second lane from the right is the negative control (wild type Bombyx mori), and the fourth and fifth lanes from the left are positive samples showing production of the tested-for A2S8 protein.

The silk product produced by transgenic Bombyx mori formed using the vectors and methods described herein produce silk with beneficially enhanced mechanical properties. In particular, conventional silkworm silk has a tensile strength of about 0.5 GPa. In contrast, spider dragline silk is often reported to have a tensile strength of about 1.1 to 1.3 GPa. Silk produced using the disclosed vectors and methods has shown tensile strength that exceeds the typical range of spider dragline silk (i.e., exceeds the range of 1.1 to 1.3 GPa). In at least one example, the produced silk exhibited a tensile strength exceeding 2.0 GPa. These results were from a transgenic silkworm that had successfully incorporated the vector shown in FIG. 1A.

Conventional silkworm silk has a breaking strain) of about 15%, whereas spider dragline silk is often reported to have a breaking strain of about 27-35%. Silk produced using the disclosed vectors and methods has demonstrated breaking strain in excess of 30%, and in at least one example, exhibiting a breaking strain exceeding 40%. Certain embodiments of the silk are thus capable of providing enhanced strength, enhanced elasticity, or both as compared to conventional silkworm silk and even in at least some instances as compared to typical dragline spider silk. These results were from a transgenic silkworm that had successfully incorporated the vector shown in FIG. 1A.

Despite these beneficial mechanical properties, the overall silk production of the transgenic Bombyx mori has not deteriorated. Tested transgenic Bombyx mori were found to maintain protein production at the same levels as their otherwise similar non-transgenic counterparts (which were the same silkworm strain subjected to transgenic modification). The ability to produce an enhanced silk product without suffering tradeoffs in overall productivity is significantly beneficial.

In certain designs, the silk product has a high proportion of spider silk. For example, in some embodiments, the silk produced by the transgenic Bombyx mori is at least about 40% spider silk proteins, or at least about 50% spider silk proteins, or at least about 60% spider silk proteins, or at least about 70% spider silk proteins, or at least about 80% spider silk proteins, or at least about 90% spider silk proteins. The high purity spider silk result from the ability to incorporate relatively large spider silk sequences and/or the ability to knockout much of the FibH gene.

### Definitions

As used herein, “modification” of the fibroin heavy chain gene includes embodiments where the entire FibH gene, or one or more portions thereof, are knocked out of the silkworm genome and replaced with a knock-in insert (e.g., using a truncation vector). The term also includes embodiments where one or more inserts are inserted at a position within the FibH gene and/or at a position functionally adjacent to the gene (e.g., using an insertion vector).

The terms “donor sequence”, “donor sequence portion”, “donor portion”, “donor insert”, and related terms are used herein to refer to the portion of a vector not including the homology arms intended to guide insertion of the vector to the target site within the FibH gene. The donor sequence may include an NTD and/or CTD sequence in addition to one or more spider protein encoding sequences. Alternatively, the donor sequence may omit the NTD and CTD sequences. The terms “vector”, “insertion vector”, are used to refer to the full sequence that includes the donor sequence and the upstream and downstream homology arms.

The terms “homologous arms” and “homology arms” are used interchangeably herein to refer to the portion of the vector intended to be homologous to a corresponding portion of the native gene on each side of the targeted location where introduction of the donor sequence is intended. Depending on where the FibH gene is targeted for nuclease activity, the NTD and CTD of the vector (if included) can act in whole or in part as homology arms.

It should be understood that the proteins and the nucleic acids encoding them may differ from the exact sequences illustrated and described herein. Thus, this disclosure includes related sequences with deletions, additions, truncations, and substitutions to the sequences shown, so long as the sequences function in accordance with the methods of the invention. Accordingly, nucleotide sequences encoding functionally equivalent variants of the illustrated sequences and proteins are included in this disclosure. For instance, changes in a DNA sequence that do not change the encoded amino acid sequence, as well as those that result in conservative substitutions of amino acid residues, one or a few amino acid deletions or additions, and/or substitution of amino acid residues by amino acid analogs are those which will not significantly affect properties of the encoded polypeptide.

Conservative amino acid substitutions include glycine/alanine; valine/isoleucine/leucine; asparagine/glutamine; aspartic acid/glutamic acid; serine/threonine/methionine; lysine/arginine; and phenylalanine/tyrosine/tryptophan. Amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. It is reasonably predictable that an isolated replacement of a leucine with an isoleucine or valine, or vice versa; an aspartate with a glutamate or vice versa; a threonine with a serine or vice versa; or a similar conservative replacement of an amino acid with a structurally related amino acid, will typically not have a major effect on activity and function of the overall protein. Proteins having substantially the same amino acid sequence as the sequences illustrated and described but possessing minor amino acid substitutions that do not substantially affect the activity or function of the protein are, therefore, within the scope of this disclosure.

Nucleotide sequences that have at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% homology or identity to the disclosed sequences may be considered functional equivalents.

Sequence identity or homology may be determined by comparing the sequences when aligned so as to maximize overlap and identity while minimizing sequence gaps. In particular, sequence identity may be determined using any of a number of mathematical algorithms. A nonlimiting example of a mathematical algorithm used for comparison of two sequences is the algorithm of Karlin & Altschul, Proc. Natl. Acad. Sci. USA 1990; 87: 2264-2268, modified as in Karlin & Altschul, Proc. Natl. Acad. Sci. USA 1993;90: 5873-5877. Another example of a mathematical algorithm used for comparison of sequences is the algorithm of Myers & Miller, CABIOS 1988;4: 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman, Proc. Natl. Acad. Sci. USA 1988; 85: 2444-2448. Advantageous for use according to the present invention is the WU-BLAST (Washington University BLAST) version 2.0 software. This program is based on WU-BLAST version 1.4, which in turn is based on the public domain NCBI-BLAST version 1.4 (Altschul & Gish, 1996, Local alignment statistics, Doolittle ed., Methods in Enzymology 266: 460-480; Altschul et al., Journal of Molecular Biology 1990; 215: 403-410; Gish & States, 1993; Nature Genetics 3: 266-272; Karlin & Altschul, 1993; Proc. Natl. Acad. Sci. USA 90: 5873-5877).

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, or less than 1% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may include properties, features (e.g., ingredients, components, members, elements, parts, and/or portions) described in other embodiments described herein. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. A method of producing transgenic Bombyx mori by targeting and modifying the FibH gene, the method comprising:
providing a gene editing assembly that includes a nuclease configured to target one or more locations within the FibH gene;
providing a vector having a donor sequence comprised of one or more spider silk sequences that each encode spider silk protein;
applying the gene editing assembly and the vector to one or more Bombyx mori cells; and
the gene editing assembly operating to incorporate the vector, including the one or more spider silk sequences, into the FibH gene,
wherein the donor sequence has a size of at least about 2 kbp.

2. The method of claim 1, wherein the donor sequence has a size of at least about 6 kbp.

3. The method of claim 1, wherein the donor sequence has a size of at least about 10 kbp.

4. The method of claim 1, wherein the gene editing assembly targets multiple locations within the FibH gene such that at least a portion of the FibH gene is knocked out and thus replaced with the donor sequence of the vector.

5. The method of claim 4, wherein at least about 50% of the FibH gene is knocked out.

6. The method of claim 1, wherein the gene editing assembly includes one or more guide RNAs (gRNAs) for targeting the one or more locations within the FibH gene, the one or more gRNAs configured to target one or more of SEQ ID NO:2 through SEQ ID NO:8.

7. The method of claim 1, wherein the gene editing assembly includes Mad7 or Cas9.

8. The method of claim 7, wherein the gene editing assembly includes Mad7, an upstream gRNA configured to target a sequence comprising one of SEQ ID NO:2 or SEQ ID NO:3, and a downstream gRNA configured to target a sequence comprising one of SEQ ID NO:4 or SEQ ID NO:5.

9. The method of claim 1, wherein the donor sequence comprises a sequence that encodes for an AS28 protein, a MaSp1 protein, a MaSp4 protein, or combination thereof.

10. The method of claim 9, wherein the donor sequence includes a sequence associated with an orb-weaver spider.

11. The method of claim 10, wherein the orb-weaver spider is Caerostris darwini.

12. The method of claim 1, wherein the donor sequence includes multiple spider silk sequences that each encode a different spider silk protein.

13. The method of claim 1, wherein the vector includes an NTD, CTD, or both.

14. The method of claim 1, wherein the vector omits an NTD and CTD.

15. The method of claim 1, wherein the donor sequence of the vector has a size more than 2 times greater than an average size of homology arms of the vector.

16. A transgenic Bombyx mori silkworm made according to the method of claim 1.

17. A silk product made by the transgenic Bombyx mori silkworm of claim 16.

18. The silk product of claim 17, wherein the silk has a tensile strength, a breaking strain, or both, that are greater than those of conventional Bombyx mori silk.

19. The silk product of claim 17, wherein the silk has a tensile strength greater than 1.1 GPa, a breaking strain in excess of 30%, or both.

20. The silk product of claim 17, wherein at least about 50% of the silk proteins are spider silk proteins.
